(19) United States
(12) Patent Application Publication
(10) Pub. No.: US 2024/0317660 A1
(43) Pub. Date: Sep. 26, 2024

(54) PROCESSES FOR THE PREPARATION OF ORTHO-ALLYLATED HYDROXY PHENYL COMPOUNDS

(71) Applicant: McMaster University, Hamilton (CA)

(72) Inventors: Jakob Magolan, Grimsby (CA); Xiong Zhang, Tianjin (CN); Nicholas Jentsch, Hamilton (CA); Mathew Piotrowski, Woodstock (CA); Meghan Fragis, Whitby (CA); Jarrod Johnson, St. George (CA); Lauren Irwin, Dundas (CA)

(73) Assignee: McMaster University, Hamilton, ON (CA)

(21) Appl. No.: 18/572,235

(22) PCT Filed: Jun. 23, 2022
(86) PCT No.: PCT/CA2022/051009

§ 371 Date: Dec. 20, 2023

Related U.S. Application Data
(60) Provisional application No. 63/202,754, filed on Jun. 23, 2021.

(51) Int. Cl. C07C 37/16

(52) U.S. Cl. CPC C07C 37/16

(57) ABSTRACT

The present application describes process for preparing an ortho-allylated hydroxy aryl compounds such as compounds of Formula (I) by reacting an allylic alcohol with a hydroxy aryl compound in the presence of aluminum compound selected from alumina and aluminum alkoxides and in a non-protic solvent wherein at least one carbon atom ortho to the hydroxy group in the hydroxy aryl compound is unsubstituted.

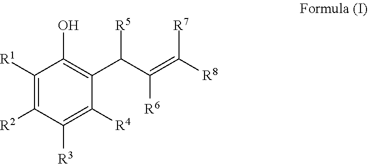

### RELATED APPLICATIONS

The present application claims the benefit of priority of co-pending U.S. provisional patent application No. 63/202,754 filed on Jun. 23, 2021 the contents of which are incorporated herein by reference in their entirety.

### FIELD

The present application is related to process for preparing ortho-allylated hydroxy phenyl compounds, in particular using alumina or aluminum alkoxides and a non-protic solvent.

### BACKGROUND

The phenol (or hydroxy aryl) moiety is ubiquitous in natural products and present in many synthetic compounds of value in medicinal chemistry and materials sciences.¹⁻³ Regiospecific ortho allylation of phenols presents a significant synthetic challenge.⁴⁻⁶ More broadly, any form of ortho-regiospecific functionalization of phenols is a synthetic challenge that has previously been addressed with creative synthetic methodology solutions.⁷⁻¹⁷

Phenols undergo electrophilic aromatic substitution (EAS) reactions with generally poor regioselectivity between ortho- and para-positions, although enhancement of ortho-selectivity has been achieved for some substrates with a variety of additives including amines⁴⁵⁻⁴⁷ ammonium salts,⁴⁸ thioureas,⁴⁹⁻⁵¹ and Lewis acids.⁵²⁻⁵⁵ Similarly, oxidative methods of phenol substitution also typically yield mixtures of ortho- and para-substituted products,⁵⁶ with ortho-enhancement reported in rare cases.⁵⁷⁻⁶¹ Ball and co-workers recently developed an ortho-specific arylation of unprotected phenols with boronic acids using a stoichiometric bismuth reagent.⁶² Therefore, the efficient one-step ortho-selective substitution unprotected phenols remains a synthetic challenge.

U.S. Pat. No. 10,059,683 describes a process for the production of cannabidiol and delta-9-tetrahydrocannabinol (and derivatives thereof) via a protic or Lewis acid-catalyzed reaction between a substituted di-halo-olivetol or derivative thereof and a suitably selected cyclic alkene. Removal of the halo groups of the resulting product is required to access the cannabidiol and delta-9-tetrahydrocannabinol compounds.

The present Applicant previously reported an efficient one step process of preparing ortho-allylated hydroxy phenyl compounds (e.g. ortho-allylated phenolics) by reaction of unprotected hydroxy phenyl compounds (e.g phenolics) with certain allylic alcohols through aluminum-promoted allylation in non-protic solvents (see PCT patent application S.N. PCT/CA2021/050733, filed May 28, 2021). In this process the C—C bond formation takes place between the ortho carbon atom of the phenol and the carbon atom to which the hydroxyl group of the allylic alcohol is attached.

### SUMMARY

The Applicant has now shown that ortho-allylated hydroxy phenyl compounds (e.g. ortho-allylated phenolics) can be accessed using an aluminum-promoted reaction between unprotected hydroxy phenyl compounds (e.g phenolics) with allylic alcohols specifically designed to allow the C—C bond formation to take place between the ortho carbon atom of the phenol and the carbon atom of the double bond that is p to the hydroxyl group of the allylic alcohol. This process opens the door to the preparation of a wide variety of useful compounds in one step from readily available precursors, including, for example, cannabidiol and derivatives thereof.

Accordingly, the application includes a process for preparing a compound of Formula (I):

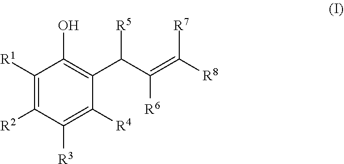

comprising reacting a compound of Formula (II):

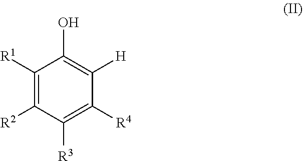

with a compound of Formula (III):

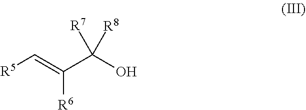

in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, alkyl, alkenyl, alkynyl, cycloalkyl, heterocycloalkyl, aryl, heteroaryl, Z-alkyl, Z-alkenyl, Z-alkynyl, Z-cycloalkyl, Z-heterocycloalkyl, Z-aryl, and Z-heteroaryl, wherein the later 14 groups are unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, SO₂-alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl;
Z is selected from O, C(O), CO₂, S, SO₂, SO, and NR¹⁰;
R⁵ is selected from H, alkyl and haloalkyl;
R⁶ is selected from H, alkyl and haloalkyl;
R⁷ is selected from alkyl, alkenyl, alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl;
R⁸ is selected from H, alkyl, alkenyl, alkynyl, aryl, heteroaryl, cycloalkyl and heterocycloalkyl, each of which is unsubstituted or substituted with one or more substituents independently selected from ═O, OH, NH₂, CN, NO₂, COOH, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl;
R⁹, R¹⁰ and R¹¹ are independently selected from H, alkyl and haloalkyl.

In an embodiment, the present application includes a process for preparing a compound of Formula (I-A):

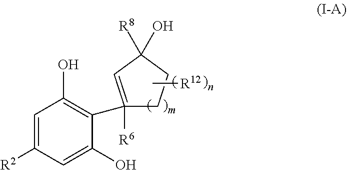

comprising reacting a compound of Formula (II):

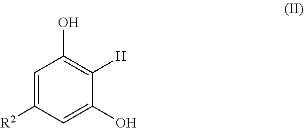

with a compound of Formula (Ill-A):

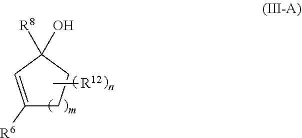

in presence of aluminum compound selected from alumina and aluminum alkoxides and in a non-protic solvent to form the compound of Formula (I-A),
wherein:
R² is selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl;
Z is selected from O, C(O), CO₂, S, SO₂, SO, and NR¹⁰;
R⁶ is selected from H, C₁₋₆alkyl and fluoroC₁₋₆alkyl;
R⁸ is selected from H, C₁₋₆alkyl and fluoroC₁₋₆alkyl;
R¹⁰ is selected from C₁₋₆alkyl and C₁₋₆fluoroalkyl;
each R¹² is independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl;
m is 1, 2, 3, 4, 5, 6, 7, or 8; and
n is 1, 2, 3 or 4.

In particular, the processes of the present application represent an efficient route to the preparation of cannabiniol (CBD), and derivatives thereof, in a single step from readily available starting materials.

The application includes a process for preparing a compound of Formula I-B

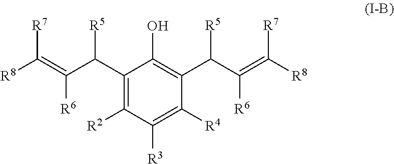

comprising reacting a compound of Formula (II):

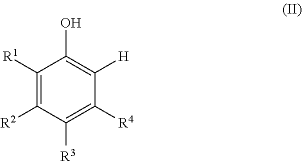

with a compound of Formula (III):

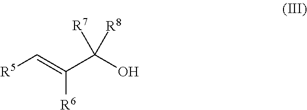

in presence of aluminum compound selected from alumina and aluminum alkoxides and in a non-protic solvent to form the compound of Formula (I),
wherein R¹, R², R³, R⁴, R⁵, R⁶, R⁷, R⁸ are as defined above for the compounds of Formula I, II and II.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

The term “process of the application” and the like as used herein refers to a process of preparing ortho-allylated hydroxy aryl compounds including compounds of Formula (I) and (I-A) as described herein.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and hydrates, as well as a combination of, for example, a solvate of a salt of a compound of the application.

As used in the present application, the singular forms “a”, “an” and “the” include plural references unless the content clearly dictates otherwise. For example, an embodiment including “a solvent” should be understood to present certain aspects with one solvent, or two or more additional solvents.

In embodiments comprising an “additional” or “second” component, such as an additional or second solvent, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. All alkyl groups are optionally fluoro-substituted.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms. All alkenyl groups are optionally fluoro-substituted.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms. All alkynyl groups are optionally fluoro-substituted.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted.

The term “alkenylene” as used herein, whether it is used alone or as part of another group, means a straight or branched chain, unsaturated alkylene group, that is, an unsaturated carbon chain that contains substituents on two of its ends and at least one double bond. The number of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally fluorosubstitutes.

The term “alkynylene” as used herein, whether it is used alone or as part of another group, means a straight or branched chain, unsaturated alkylene group, that is, an unsaturated carbon chain that contains substituents on two of its ends and at least one triple bond. The number of carbon atoms that are possible in the referenced alkynylene group are indicated by the prefix “Cₙ₁₋ₙ₂”. For example, the term C₂₋₆alkynylene means an alkynylene group having 2, 3, 4, 5 or 6 carbon atoms. All alkynylene groups are optionally fluorosubstituted.

The term “aryl” as used herein, whether it is used alone or as part of another group, refers to cyclic groups containing from 6 to 20 atoms and at least one carbocyclic aromatic ring. All aryl groups are optionally fluorosubstituted.

The term “cycloalkyl,” as used herein, whether it is used alone or as part of another group, refers to cyclic groups containing from 3 to 20 atoms and at least one carbocyclic non-aromatic ring. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms. All cycloalkyl groups are optionally fluoro-substituted.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. All heterocycloalkyl groups are optionally fluoro-substituted. The heteroatom in heterocycloalkyl groups is optionally substituted or oxidized where valency allows.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. All heteroaryl groups are optionally fluoro-substituted. The heteroatom in heteroaryl groups is optionally substituted or oxidized where valency allows.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond. All cyclic groups are optionally fluoro-substituted.

The term “ring system” as used herein refers to a carbon- or heteroatom-containing ring system, that includes monocycles, fused bicyclic and polycyclic rings, and bridged rings.

The term “polycyclic” as used herein means cyclic groups that contain more than one ring linked together and includes, for example, groups that contain two (bicyclic), three (tricyclic) or four (quadracyclic) rings. The rings may be linked through a single bond, a single atom (spirocyclic) or through two atoms (fused and bridged). All polycyclic groups are optionally fluoro-substituted.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo-substituted” as used herein, refers to the substitution of one or more, including all, available hydrogens in a referenced group with halo.

The term “haloalkyl” as used herein, refers to the substitution of one or more, including all, available hydrogens in an alkyl group with halo.

The term “fluoroalkyl” as used herein, refers to the substitution of one or more, including all, available hydrogens in an alkyl group with fluoro.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a substituent.

The term “protecting group” or “PG” or “protected” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “allylic alcohol” as used herein refers to a compound comprising a hydroxy substituent (—OH) attached to a sp³ hybridized carbon which is adjacent to a double bond.

The term “hydroxy aryl compound(s)” or “phenolics” as used herein refers to a compound comprising at least one hydroxy substituent on an aryl ring. In the case of phenolics, the aryl group is phenyl.

The term “ortho-allylated hydroxy aryl compound(s)” or “ortho-allylated hydroxy phenolics” as used herein refers to a compound comprising an allylic group in a position ortho to a hydroxy group on an aryl ring. In the case of ortho-allylated hydroxy phenolics, the aryl group is phenyl.

The term “allylic group” as used herein refer to a substituent comprising a double bond adjacent to a methylene which is covalently attached to the rest of the molecule.

The term “alumina” as used herein refers to aluminium oxide having the chemical formula: Al₂O₃(H₂O)ₙ where n is in the range of 0 to 1.

The term “acidic alumina” as used herein refers to activated alumina that has been treated so that a 5% aqueous suspension of the alumina has a pH less than 7.

The term “basic alumina” as used herein refers to activated alumina that has been treated so that a 5% aqueous suspension of the alumina has a pH of greater than 7.

The term “neutral alumina” as used herein refers to activated alumina wherein a 5% aqueous suspension of the alumina has a neutral pH.

The term “activated alumina” as used herein refers to alumina that has been treated under dehydroxylation conditions to provide a highly porous material with a low water content.

The term “aluminum alkoxide” as used herein refers to a compound having having one to three reactive alkoxy (—O-alkyl) groups per atom of aluminum.

The term “aluminum isopropoxide” as used herein refers to a compound having one to three reactive isopropoxy groups per atom of aluminum.

The term “non-protic solvent” as used herein includes both non polar solvent and polar aprotic solvents.

The term “non-polar solvent” as used herein refers to a solvent that has little or no polarity and includes hydrophobic solvents.

The term “polar aprotic solvent” as used herein refers to a solvent a solvent that does not have an acidic proton and is polar.

The term “including the atoms in the phenyl ring to which said R¹, R², R³ and R⁴ groups are bonded” as used herein means that the specified number of atoms in the polycyclic ring system includes the 6 carbon atoms in the phenyl ring.

The term “unsubstituted”, as used herein means that the referenced atom does not contain a substituent group other than a hydrogen atom.

The term “substituted” as used herein means that the referenced atom contains at least one substituent group other that a hydrogen atom.

The term “substituent group” as used herein refers to any chemical grouping, including groups comprising carbon atoms and/or heteroatoms, that is compatible with the reaction conditions of the processes of the application.

The term “major isomer” as used herein refers to a stereochemical isomer, including a regional isomer, that is the most abundant isomer in a mixture of isomers of the same compound. Conversely, the term “minor isomer” as used herein refers to a stereochemical isomer, including a regional isomer, that is not the most abundant isomer in a mixture of isomers of the same compound.

In the processes of the application, it is typical for the compounds, including starting materials and products to be present as a mixture of isomers. For example, when it is shown that the R- or S-isomer is a product or starting material of a reaction, this means that that isomer is present in greater than 80%, 85%, 90%, 95%, 98% or 99% by weight based on the total amount of R- and S-isomers.

The products of the processes of the application may be isolated according to known methods, for example, the compounds may be isolated by evaporation of the solvent, by filtration, centrifugation, chromatography or other suitable method.

### II. Processes of the Application

The present application is directed to a process for preparing a compound of Formula (I):

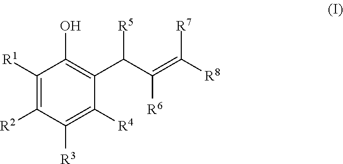

comprising reacting a compound of Formula (II):

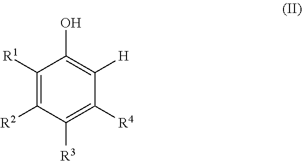

with a compound of Formula (III):

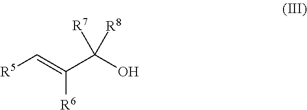

in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, alkyl, alkenyl, alkynyl, cycloalkyl, heterocycloalkyl, aryl, heteroaryl, Z-alkyl, Z-alkenyl, Z-alkynyl, Z-cycloalkyl, Z-heterocycloalkyl, Z-aryl, and Z-heteroaryl, wherein the later 14 groups are unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, SO₂-alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, haloalkyl, alkenyl, Oalkyl and Ohaloalkyl;
Z is selected from O, C(O), CO₂, S, SO₂, SO, and NR¹⁰;
R⁵ is selected from H, alkyl and haloalkyl;
R⁶ is selected from H, alkyl and haloalkyl;
R⁷ is selected from alkyl, alkenyl, alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl;
R⁸ is selected from H, alkyl, alkenyl, alkynyl, aryl, heteroaryl, cycloalkyl and heterocycloalkyl, each of which is unsubstituted or substituted with one or more substituents independently selected from ═O, OH, NH₂, CN, NO₂, COOH, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl haloalkyl, alkenyl, Oalkyl and Ohaloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl haloalkyl, alkenyl, Oalkyl and Ohaloalkyl; R⁹, R¹⁰ and R¹¹ are independently selected from H, alkyl and haloalkyl.

The present application is directed to a process for preparing a compound of Formula (I):

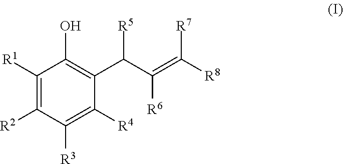

comprising reacting a compound of Formula (II):

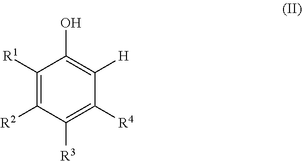

with a compound of Formula (III):

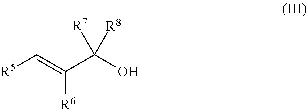

in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, alkyl, alkenyl, alkynyl, cycloalkyl, heterocycloalkyl, aryl, heteroaryl, Z-alkyl, Z-alkenyl, Z-alkynyl, Z-cycloalkyl, Z-heterocycloalkyl, Z-aryl, and Z-heteroaryl, wherein the later 14 groups are unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, SO₂-alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, haloalkyl, alkenyl, Oalkyl and Ohaloalkyl;
Z is selected from O, C(O), CO₂, S, SO₂, SO, and NR¹⁰;
R⁵ is selected from H, alkyl and haloalkyl;
R⁶ is selected from H, alkyl and haloalkyl;
R⁷ is selected from alkyl, alkenyl, alkynyl, aryl, heteroaryl, cycloalkyl and heterocycloalkyl, each of which is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl, haloalkyl, alkenyl, Oalkyl and Ohaloalkyl;
R⁸ is selected from H, alkyl, alkenyl, alkynyl, aryl, heteroaryl, cycloalkyl and heterocycloalkyl, each of which is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl haloalkyl, alkenyl, Oalkyl and Ohaloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHalkyl, N(alkyl)(alkyl), alkyl, alkenyl, alkynyl, Oalkyl, Oalkenyl, Oalkynyl, Salkyl, Salkenyl, Salkynyl, S(O)alkyl, S(O)alkenyl, S(O)alkynyl, SO₂alkyl, SO₂alkenyl, SO₂alkynyl, aryl, heteroaryl, cycloalkyl, and heterocycloalkyl, the latter 21 groups being unsubstituted or further substituted with one or more substituents independently selected from OH, halo, alkyl haloalkyl, alkenyl, Oalkyl and Ohaloalkyl;
R⁹, R¹⁰ and R¹¹ are independently selected from H, alkyl and haloalkyl.

In an embodiment, the application further includes a process for preparing a compound of Formula (I):

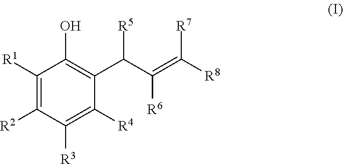

comprising reacting a compound of Formula (II):

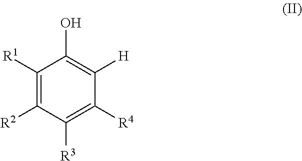

with a compound of Formula (III):

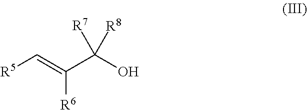

in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, C₁₋₂₀alkyl, C₁₋₂₀haloalkyl, C₂₋₂₀alkenyl, C₂₋₂₀alkynyl, C₃₋₂₀cycloalkyl, C₃₋₂₀heterocycloalkyl, aryl, C₅₋₂₀heteroaryl, Z—C₁₋₂₀alkyl, Z—C₂₋₂₀alkenyl, Z—C₂₋₂₀alkynyl, Z—C₃₋₂₀cycloalkyl, Z—C₃₋₂₀heterocycloalkyl, Z-aryl, and Z—C₅₋₂₀heteroaryl, wherein the later 14 groups are unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; Z is selected from O, C(O), CO₂, S, SO₂, SO, and NR¹⁰;
R⁵ is selected from H, C₁₋₆alkyl and C₁₋₆haloalkyl;
R⁶ is selected from H, C₁₋₆alkyl and C₁₋₆haloalkyl;
R⁷ is selected from C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁸ is selected from H, C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁹, R¹⁰ and R¹¹ are independently selected from H, C₁₋₆alkyl and C₁₋₆haloalkyl.

In an embodiment, the application further includes a process for preparing a compound of Formula (I):

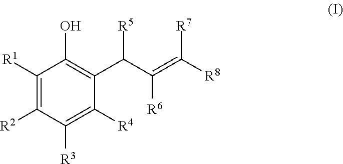

comprising reacting a compound of Formula (II):

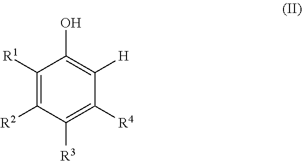

with a compound of Formula (III):

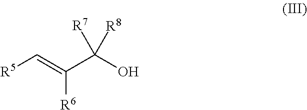

in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, C₁₋₂₀alkyl, C₁₋₂₀haloalkyl, C₂₋₂₀alkenyl, C₂₋₂₀alkynyl, C₃₋₂₀cycloalkyl, C₃₋₂₀heterocycloalkyl, aryl, C₅₋₂₀heteroaryl, Z—C₁₋₂₀alkyl, Z—C₂₋₂₀alkenyl, Z—C₂₋₂₀alkynyl, Z—C₃₋₂₀cycloalkyl, Z—C₃₋₂₀heterocycloalkyl, Z-aryl, and Z—C₅₋₂₀heteroaryl, wherein the later 14 groups are unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
Z is selected from O, C(O), CO₂, S, SO₂, SO, and NR¹⁰;
R⁵ is selected from H, C₁₋₆alkyl and C₁₋₆haloalkyl;
R⁶ is selected from H, C₁₋₆alkyl and C₁₋₆haloalkyl;
R⁷ is selected from C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁸ is selected from H, C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, NHC₁₋₁₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁹, R¹⁰ and R¹¹ are independently selected from H, C₁₋₆alkyl and C₁₋₆haloalkyl.

In an embodiment, R¹, R², R³ and R⁴ are independently selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

In an embodiment, R¹, R², R³ and R⁴ are independently selected from H, OH, F, Cl, Br, CN, NO₂, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₂₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

In an embodiment, R¹, R², R³ and R⁴ are independently selected from H, OH, F, Cl, Br, CN, NO₂, C₁₋₁₀alkyl, C₁₋₁₀haloalkyl, C₂₋₁₀alkenyl, Z—C₁₋₁₀alkyl and Z—C₂₋₁₀alkenyl, wherein the later 5 groups are unsubstituted or substituted with one or more substituents independently selected from OH, NH₂, halo, NHC₁₋₁₀alkyl, N(C₁₋₆alkyl)(C₁₋₆ alkyl), C₁₋₆alkyl, C₂₋₆alkenyl, OC₁₋₆alkyl, OC₂₋₆alkenyl, OC₂₋₆alkynyl, SC₁₋₆alkyl and SC₂₋₆alkenyl; the latter 9 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl.

In an embodiment, R¹, R², R³ and R⁴ are independently selected from H, OH, F, Cl, Br, CN, NO₂, C₁₋₁₀alkyl, C₁₋₁₀haloalkyl, C₂₋₁₀alkenyl, Z—C₁₋₁₀alkyl and Z—C₂₋₁₀alkenyl.

In an embodiment, R¹ is selected from H, F, Cl, Br, CN, NO₂, C₁₋₁₀alkyl, C₁₋₁₀haloalkyl and Z—C₁₋₁₀alkyl.

In an embodiment, R¹ is C₂₋₁₀alkenyl which is unsubstituted or substituted with one or more substituents independently selected from OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl. In an embodiment, R¹ is C₂₋₁₀alkenyl which is unsubstituted or substituted with one or more substituents independently selected from OH, NH₂, F, Cl, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl. In an embodiment, R¹ is C₂₋₁₀alkenyl which is unsubstituted or substituted with one or more substituents independently selected from OH, NH₂, F, Cl, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SO₂C₁₋₆alkyl, aryl, C₅₋₁₀heteroaryl, C₃₋₁₀cycloalkyl and C₃₋₁₀heterocycloalkyl, the latter 14 groups being unsubstituted or further substituted with one or more substituents independently selected from OH, halo, C₁₋₄alkyl, C₁₋₄haloalkyl, C₂₋₄alkenyl, OC₁₋₄alkyl and OC₁₋₄haloalkyl.

In an embodiment, R² is selected from H, F, Cl, Br, CN, NO₂, C₁₋₁₀alkyl, C₁₋₁₀haloalkyl, C₂₋₁₀alkenyl and Z—C₁₋₁₀alkyl. In an embodiment, R² is selected from H, F, Cl, Br, NO₂, C₁₋₁₀alkyl, C₁₋₁₀haloalkyl and Z—C₁₋₁₀alkyl. In an embodiment, R² is selected from C₁₋₁₀alkyl, C₁₋₁₀haloalkyl and Z—C₁₋₁₀alkyl. In an embodiment, R² is Z—C₁₋₁₀alkyl. In an embodiment, R² is C₁₋₁₀alkyl. In an embodiment, R² is selected from C₁alkyl, C₃alkyl, C₅alkyl and C₇alkyl.

In an embodiment, R³ is selected from H, F, Cl, Br, CN, NO₂, C₁₋₁₀alkyl, C₁₋₁₀haloalkyl and Z—C₁₋₁₀alkyl.

In an embodiment, R⁴ is selected from H, OH, F, Cl, Br, CN, NO₂, C₁₋₁₀alkyl, C₁₋₁₀haloalkyl and Z—C₁₋₁₀alkyl. In an embodiment, R⁴ is selected from H, OH, C₁₋₁₀alkyl, C₁₋₁₀haloalkyl and Z—C₁₋₁₀alkyl. In an embodiment, R⁴ is selected from H, OH, C₁₋₁₀alkyl, and Z—C₁₋₁₀alkyl. In an embodiment, R⁴ is C₁₋₄alkyl. In an embodiment, R⁴ is Z—C₁₋₄alkyl.

In an embodiment, R⁴ is selected from H and OH. In an embodiment, R⁴ is OH.

In an embodiment, Z is selected from S, SO₂ and SO.

In an embodiment Z is selected from O, C(O) and CO₂.

In an embodiment, Z is NR¹⁰.

In an embodiment, Z is selected from SO₂, O, C(O) and NR¹⁰. In an embodiment, Z is selected from O and C(O). In an embodiment, Z is C(O). In an embodiment, Z is O.

In an embodiment, R¹⁰ is selected from H, C₁₋₆alkyl and C₁₋₆fluoroalkyl. In an embodiment, R¹⁰ is selected from H, C₁₋₄alkyl and C₁₋₄fluoroalkyl. In an embodiment, R¹⁰ is H.

In an embodiment, when R¹, R², R³ and R⁴ are independently selected from Z—C₁₋₁₀alkyl and Z—C₂₋₁₀alkenyl, Z is selected from O and C(O). In an embodiment, when R¹, R², R³ and R⁴ are independently selected from Z—C₁₋₁₀alkyl, Z is selected from O and C(O). In an embodiment, when R¹, R², R³ and R⁴ are independently selected from Z—C₁₋₁₀alkyl, Z is O.

In an embodiment, the compound of Formula (II) is selected from

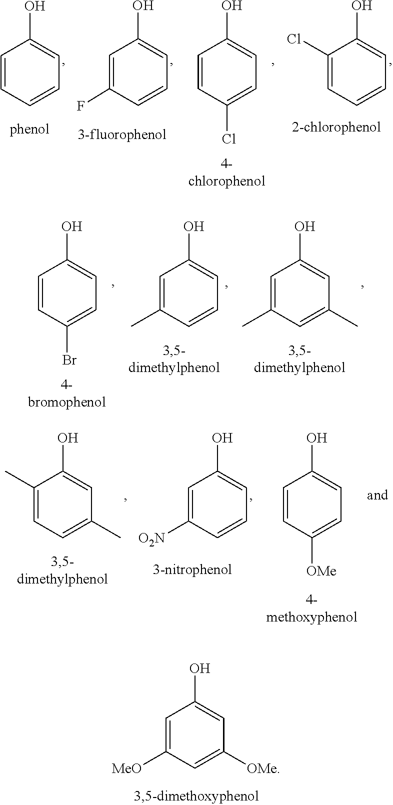

In an embodiment, when R⁴ is OH, the compound of Formula (II) is

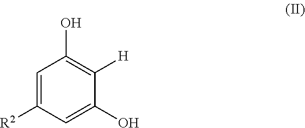

wherein:
R² is selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl;
Z is selected from O, C(O), CO₂, S, SO₂, SO, and NR¹⁰; and
R¹⁰ is selected from H, C₁₋₆alkyl and C₁₋₆fluoroalkyl.

In an embodiment, when R⁴ is OH, R² in the compound of Formula (II) is selected from H, OH, halo, CN, NO₂, C₁₋₁₀alkyl, C₁₋₁₀haloalkyl, C₂₋₁₀alkenyl, Z—C₁₋₁₀alkyl and Z—C₂₋₁₀alkenyl, wherein the later 5 groups are unsubstituted or substituted with one or more substituents independently selected from OH, NH₂, halo, NHC₁₋₆alkyl, N(C₁₋₆alkyl)(C₁. 6alkyl), C₁₋₆alkyl, C₂₋₆alkenyl, OC₁₋₆alkyl, OC₂₋₆alkenyl, OC₂₋₆alkynyl, SC₁₋₆alkyl and SC₂₋₆alkenyl; the latter 9 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl. In an embodiment, when R⁴ is OH, R² in the compound of Formula (II) is selected from H, halo, OH, C₁₋₁₀alkyl, C₁₋₁₀fluoroalkyl, C₂₋₁₀alkenyl and Z—C₁₋₁₀alkyl.

In an embodiment, when R⁴ is OH and R² in the compound of Formula (II) is selected from Z—C₁₋₁₀alkyl and Z—C₂₋₁₀alkenyl, Z is selected from O and C(O). In an embodiment, when R⁴ is OH and R² is Z—C₁₋₁₀alkyl, Z is selected from O and C(O). In an embodiment, when R⁴ is OH and R² is Z—C₁₋₁₀alkyl, Z is O.

In an embodiment, R² is selected from H, halo, OH, C₁₋₁₀alkyl, C₁₋₁₀fluoroalkyl and C₂₋₁₀alkenylln an embodiment, when R⁴ is OH, R² in the compound of Formula (II) is selected from H, F, Cl, Br, NO₂, C₁₋₁₀alkyl, C₁₋₁₀haloalkyl and Z—C₁₋₁₀alkyl. In an embodiment, when R⁴ is OH, R² in the compound of Formula (II) is C₁₋₁₀alkyl. In an embodiment, when R⁴ is OH, R² in the compound of Formula (II) is selected from C₁alkyl, C₃alkyl, C₅alkyl and C₇alkyl.

In an embodiment, when R⁴ is OH, the compound of Formula (II) is selected from

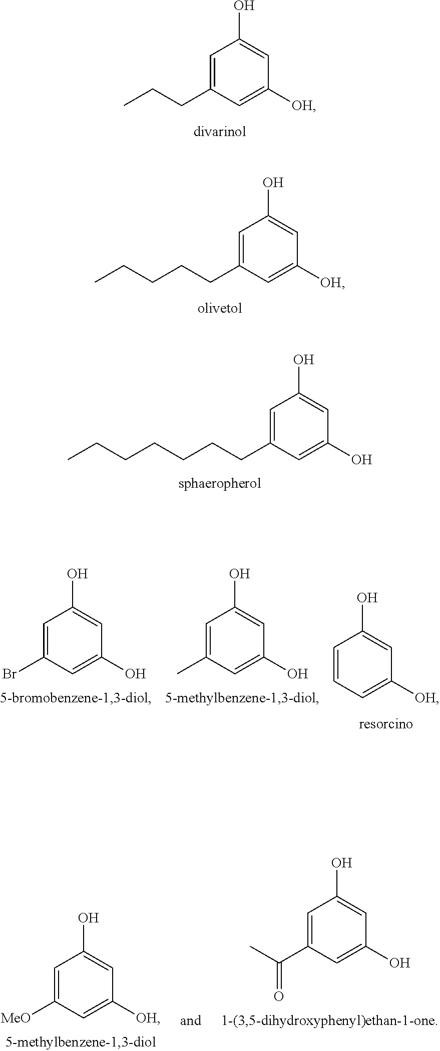

In an embodiment the compound of Formula (II) is selected from

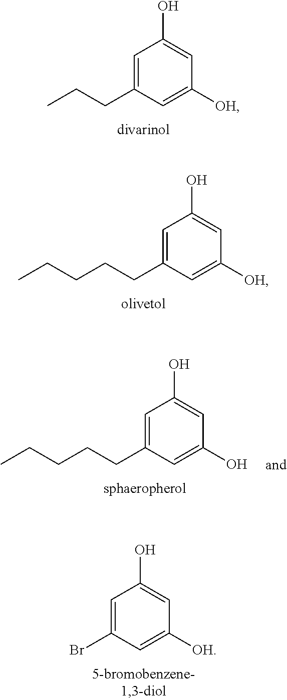

In an embodiment, R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

In an embodiment, R¹ and R², R² and R³ and/or R³ and R⁴ are linked together to form a polycyclic ring system having 10 or more atoms including the atoms in the phenyl ring to which said R¹, R², R³ and R⁴ groups are bonded, and in which one to three carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl. In an embodiment, R³ and R⁴ are linked together to form a polycyclic ring system having 10 or more atoms including the atoms in the phenyl ring to which said R³ and R⁴ groups are bonded, and in which one to three carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

In an embodiment, R⁹ is selected from H, C₁₋₄alkyl and C₁₋₄haloalkyl.

In an embodiment, R³ and R⁴ are linked together to form a polycyclic ring system having 10 or more atoms including the atoms in the phenyl ring to which said R³ and R⁴ groups are bonded, and the polycyclic ring system is unsubstituted or substituted with one or more substituents independently selected from ═O, OH, NH₂, halo, NHC₁₋₂₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

Therefore in an embodiment, when R³ and R⁴ are linked together to form a polycyclic ring system, the compound of Formula (II) has the following structure:

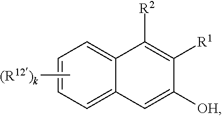

wherein:
R¹ is selected from H, C₁₋₆alkyl and fluoroC₁₋₆alkyl;
R² is selected from H, C₁₋₆alkyl and fluoroC₁₋₆alkyl;
each R^{12′} is independently selected from OH, NH₂, halo, NO₂, CN, COOH, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl; and
k is 0, 1, 2, 3 or 4.

In an embodiment, when R³ and R⁴ are linked together to form a polycyclic ring system, R¹ is selected from H, C₁₋₄alkyl and fluoroC₁₋₄alkyl. In an embodiment, when R³ and R⁴ are linked together to form a polycyclic ring system, R¹ is H.

In an embodiment, when R³ and R⁴ are linked together to form a polycyclic ring system, R² is selected from H, C₁₋₄alkyl and fluoroC₁₋₄alkyl. In an embodiment, when R³ and R⁴ are linked together to form a polycyclic ring system, R² is H.

In an embodiment, each R^{12′} is independently selected from OH, NH₂, halo, NO₂, CN, COOH, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₁₋₆fluoroalkyl, OC₂₋₆alkenyl, OC₂₋₆alkynyl, SC₁₋₆alkyl, SC₁₋₆fluoroalkyl SC₂₋₆alkenyl, SC₂₋₆alkynyl, S(O)C₁₋₆alkyl, S(O)C₁₋₆fluoroalkyl, S(O)C₂₋₆alkenyl, S(O)C₂₋₆alkynyl, SO₂C₁₋₆alkyl, SO₂C₁₋₆fluoroalkyl. SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₄alkyl, C₁₋₄fluoroalkyl C₂₋₄alkenyl, OC₁₋₄alkyl and OC₁₋₄fluoroalkyl.

In an embodiment, k is 0, 1 or 2. In an embodiment, k is 0.

In an embodiment, when R³ and R⁴ are linked together to form a polycyclic ring system, the compound of Formula (II) is

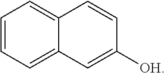 [SMILES: Oc1ccc2ccccc2c1]

In an embodiment, R⁵ and R⁷ in the compound of Formula (III) are linked together to form an unsubstituted or substituted monocyclic ring. Therefore in an embodiment, the compound of Formula (III) has the following structure:

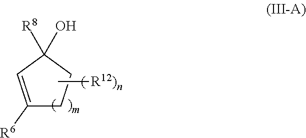

wherein:
R⁸ is selected from H, C₁₋₆alkyl and fluoroC₁₋₆alkyl; R⁶ is selected from H, C₁₋₆alkyl and fluoroC₁₋₆alkyl;
each R¹² is independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀ heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl;
m is 1, 2, 3, 4, 5, 6, 7, or 8; and
n is 1, 2, 3 or 4.

In an embodiment, m is 2 and n is 1 or m is 4 and n is 1 and the compound of Formula (III-A) is selected from:

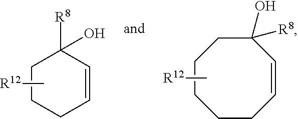

wherein:
R⁸ is selected from H, C₁₋₆alkyl and fluoroC₁₋₆alkyl; and
R¹² is selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl.

In an embodiment, R¹² is C₂₋₁₀alkenyl which is unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl. In an embodiment, R¹² is C₂₋₁₀alkenyl which is unsubstituted or further substituted with one or more substituents independently selected from OH, halo, C₁₋₄alkyl, C₁₋₄fluoroalkyl, C₂₋₄alkenyl, OC₁₋₄alkyl and OC₁₋₄fluoroalkyl. In an embodiment, R¹² is C₂₋₆alkenyl substituted with C₁₋₄alkyl. In an embodiment, R¹² is

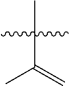

wherein [symbol] is a point of attachment to compound of Formula (III-A). In an embodiment, R¹² is C₂₋₆alkenyl substituted with halo. Therefore, in embodiment, R¹² is haloC₂₋₆alkenyl. In an embodiment, R¹² is fluoroC₂₋₆alkenyl.

In an exemplary embodiment, the compound of Formula (III-A) is selected from

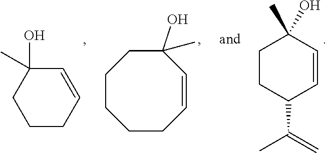

In an embodiment, the compound of Formula (III) has the following structure:

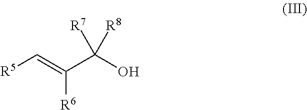

wherein:
R⁵ is selected from H, C₁₋₆alkyl and C₁₋₆haloalkyl;
R⁶ is selected from H, C₁₋₆alkyl and C₁₋₆haloalkyl;
R⁷ is selected from C₁₋₂₀alkyl, C₂₋₂₀alkenyl, C₂₋₂₀alkynyl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁸ is selected from H, C₁₋₂₀alkyl, C₂₋₂₀alkenyl, C₂₋₂₀alkynyl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁹, R¹⁰ and R¹¹ are independently selected from H, C₁₋₆alkyl and C₁₋₆haloalkyl.

In an embodiment, the compound of Formula (III) has the following structure:

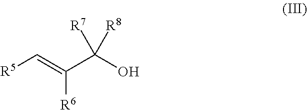

wherein:
R⁵ is selected from H, C₁₋₆alkyl and C₁₋₆haloalkyl;
R⁶ is selected from H, C₁₋₆alkyl and C₁₋₆haloalkyl;
R⁷ is selected from C₁₋₂₀alkyl, C₂₋₂₀alkenyl, C₂₋₂₀alkynyl, cycloalkyl and heterocycloalkyl, each of which is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁸ is selected from H, C₁₋₂₀alkyl, C₂₋₂₀alkenyl, C₂₋₂₀alkynyl, cycloalkyl and heterocycloalkyl, each of which is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀ haloalkyl;
R⁹, R¹⁰ and R¹¹ are independently selected from H, C₁₋₆alkyl and C₁₋₆haloalkyl.

In an embodiment, R⁵ is selected from H, C₁₋₃alkyl and C₁₋₃haloalkyl. In an embodiment, R⁵ is selected from H, CH₃ and CF₃. In an embodiment, R⁵ is H.

In an embodiment, R⁶ is selected from H, C₁₋₃alkyl and C₁₋₃haloalkyl. In an embodiment, R⁶ is selected from H, CH₃ and CF₃. In an embodiment, R⁶ is H.

In an embodiment, R⁷ is selected from C₁₋₁₀alkyl, C₂₋₁₀alkenyl, C₂₋₁₀alkynyl, aryl, C₅₋₁₀heteroaryl, C₃₋₁₀cycloalkyl and C₃₋₁₀heterocycloalkyl each of which is unsubstituted or substituted with one or more substituents independently selected from ═O, OH, NH₂, halo, CN, NO₂, COOH, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl. In an embodiment, R⁷ is selected from C₁₋₁₀alkyl, C₂₋₁₀alkenyl, C₂₋₁₀alkynyl, aryl, C₅₋₁₀heteroaryl, C₃₋₁₀cycloalkyl and C₃₋₁₀heterocycloalkyl each of which is unsubstituted or substituted with one or more substituents independently selected from OH, NH₂, halo, CN, NO₂, COOH, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl. In an embodiment, R⁷ is selected from C₁₋₁₀alkyl, C₂₋₁₀alkenyl, aryl, C₅₋₁₀heteroaryl, C₃₋₁₀cycloalkyl and C₃₋₁₀heterocycloalkyl each of which is unsubstituted or substituted with one or more substituents independently selected from OH, NH₂, F, Cl, CN, NO₂, COOH, NHC₁₋₆alkyl, N(C₁₋₆alkyl)(C₁₋₆alkyl), C₁₋₆alkyl, C₂₋₆alkenyl, OC₁₋₆alkyl, OC₂₋₆alkenyl. In an embodiment, R⁷ is selected from C₁₋₁₀alkyl, C₂₋₁₀alkenyl, aryl, C₅₋₁₀heteroaryl, C₃₋₁₀cycloalkyl and C₃₋₁₀heterocycloalkyl each of which is unsubstituted or substituted with one or more substituents independently selected from OH, NH₂, F, Cl, CN, NO₂, COOH, NHC₁₋₆alkyl, N(C₁₋₆alkyl)(C₁₋₆alkyl), C₁₋₆alkyl and OC₁₋₁₀alkyl.

In an embodiment, R⁷ is C₁₋₁₀alkyl.

In an embodiment, R⁷ is C₂₋₁₀alkenyl which is unsubstituted or substituted with one or more substituents independently selected from OH, NH₂, F, Cl, CN, NO₂, COOH, NHC₁₋₆alkyl, N(C₁₋₆alkyl)(C₁₋₆alkyl), C₁₋₆alkyl and OC₁₋₁₀alkyl.

In an embodiment, R⁷ is C₃₋₇cycloalkyl which is unsubstituted or substituted with one or more substituents independently selected from OH, NH₂, F, Cl, CN, NO₂, COOH, NHC₁₋₆alkyl, N(C₁₋₄alkyl)(C₁₋₄alkyl), C₁₋₄alkyl and OC₁₋₄alkyl.

In an embodiment, R⁷ is aryl or C₅₋₁₀heteroaryl which are unsubstituted or substituted with one or more substituents independently selected from OH, NH₂, F, Cl, CN, NO₂, COOH, NHC₁₋₆alkyl, N(C₁₋₄alkyl)(C₁₋₄alkyl), C₁₋₄alkyl and OC₁₋₄alkyl.

In an embodiment, R⁸ is selected from H, C₁₋₁₀alkyl and C₂₋₂₀alkenyl, each of which is unsubstituted or substituted with one or more substituents independently selected from OH, NH₂, F, Cl, NHC₁₋₁₀alkyl, N(C₁₋₁₀alkyl)(C₁₋₁₀alkyl), C₁₋₁₀alkyl, C₂₋₁₀alkenyl, , OC₁₋₁₀alkyl and OC₂₋₁₀alkenyl. In an embodiment, R⁸ is selected from H and C₁₋₆alkyl which is unsubstituted or substituted with one or more substituents independently selected from OH, NH₂, F, Cl, NHC₁₋₄alkyl, N(C₁₋₄alkyl)(C₁₋₄alkyl), C₁₋₄alkyl, C₂₋₄alkenyl, OC₁₋₄alkyl and OC₂₋₄alkenyl.

In an embodiment, any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;

In an embodiment, R⁷ and R⁸ in the compound of Formula (III) are linked together to form an unsubstituted or substituted monocyclic ring in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl.

In an embodiment, R¹¹ is selected from H, C₁₋₄alkyl and C₁₋₄haloalkyl.

In an embodiment, R⁷ and R⁸ in the compound of Formula (III) are linked together to form an unsubstituted or substituted monocyclic ring which is unsubstituted or substituted with one or more substituents independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl.

Therefore in an embodiment, the compound of Formula (III) has the following structure:

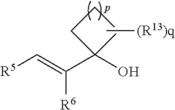

wherein
each R¹³ is independently selected from ═O, OH, NH₂, halo, CN, NO₂, COOH, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl;
p is 1, 2, 3, 4 or 5 and
q is 0, 1, 2, 3 or 4.

In an embodiment, each R¹³ is independently selected ═O, OH, NH₂, halo, CN, NO₂, COOH, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₁₋₆fluoroalkyl, OC₂₋₆alkenyl, OC₂₋₆alkynyl, SC₁₋₆alkyl, SC₁₋₆fluoroalkyl, SC₂₋₆alkenyl, SC₂₋₆alkynyl, S(O)C₁₋₆alkyl, S(O)C₁₋₆fluoroalkyl, S(O)C₂₋₆alkenyl, S(O)C₂₋₆alkynyl, SO₂C₁₋₆alkyl, SO₂C₁₋₆fluoroalkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₄alkyl, C₁₋₄fluoroalkyl, C₂₋₄alkenyl, OC₁₋₄alkyl and OC₁₋₄fluoroalkyl;

In an embodiment, q is 0, 1 or 2.

In an embodiment, the compound of Formula (III) is selected from

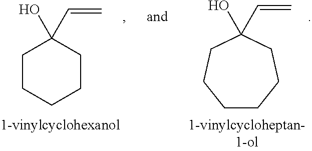

In an embodiment, when both R⁵ and R⁶ are H, the compound of Formula (III) has the following structure:

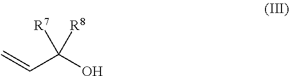

wherein:
R⁷ is selected from C₁₋₁₀alkyl, C₂₋₁₀alkenyl, C₂₋₁₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₆ alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl; and
R⁸ is selected from H, C₁₋₆alkyl and fluoroC₁₋₆alkyl.

In an embodiment the compound of Formula (III) has the following structure:

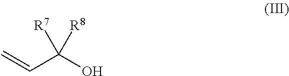

wherein:
R⁷ is selected from C₁₋₁₀alkyl, C₂₋₁₀alkenyl, C₂₋₁₀alkynyl, aryl, heteroaryl, cycloalkyl and heterocycloalkyl, each of which is unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl; and
R⁸ is selected from H, C₁₋₆alkyl and fluoroC₁₋₆alkyl.

In an embodiment, when both R⁵ and R⁶ are H, the compound of Formula (III) has the following structure:

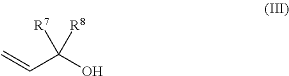

wherein:
R⁷ is selected from C₁₋₁₀alkyl, C₂₋₁₀alkenyl, C₂₋₁₀alkynyl, cycloalkyl and heterocycloalkyl, each of which is unsubstituted or substituted with one or more substituents independently selected from ═O, OH, NH₂, halo, CN, NO₂, COOH, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl; and
R⁸ is selected from H, C₁₋₆alkyl and fluoroC₁₋₆alkyl.

In an embodiment the compound of Formula (III) has the following structure:

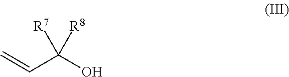

wherein:
R⁷ is selected from C₁₋₁₀alkyl, C₂₋₁₀alkenyl, C₂₋₁₀alkynyl, cycloalkyl and heterocycloalkyl, each of which is unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl; and
R⁸ is selected from H, C₁₋₆alkyl and fluoroC₁₋₆alkyl.

In an embodiment, R⁷ is selected from C₁₋₁₀alkyl, C₂₋₁₀alkenyl, C₂₋₁₀alkynyl, C₃₋₂₀cycloalkyl, and C₃₋₂₀heterocycloalkyl, each of which is unsubstituted or substituted with one or more substituents independently selected from ═O, OH, NH₂, halo, CN, NO₂, COOH, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl.

In an embodiment, R⁷ is selected from C₁₋₁₀alkyl, C₂₋₁₀alkenyl, C₂₋₁₀alkynyl, C₃₋₁₀cycloalkyl and C₃₋₁₀heterocycloalkyl, each of which is unsubstituted or substituted with one or more substituents independently selected from ═O, OH, NH₂, halo, CN, NO₂, COOH NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₆alkenyl, OC₁₋₆alkyl and OC₁₋₆fluoroalkyl.

In an embodiment, R⁷ is C₁₋₁₀alkyl. In an embodiment, R⁷ is C₁₋₄alkyl. In an embodiment, R⁷ is CH₃, CH₂CH₃, CH(CH₃)₂, and CH₂CH₂CH₃. In an embodiment, R⁷ is CH₃.

In an embodiment, R⁷ is C₂₋₁₀alkenyl which is unsubstituted or substituted with one or more substituents independently selected from NH₂, F, Cl, CN, NO₂, COOH NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl. In an embodiment, R⁷ is C₂₋₁₀alkenyl which is unsubstituted or substituted with one or more substituents independently selected from F, Cl, C₁₋₆alkyl and C₂₋₆alkenyl. In an embodiment, R⁷ is C₂₋₁₀alkenyl which is unsubstituted or substituted with one or more substituents independently selected from C₁₋₆alkyl and C₂₋₆alkenyl.

In an embodiment, R⁷ a comprises a prenyl functional group or repeating prenyl functional groups. In an embodiment, R⁷ is

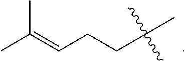 [SMILES: *CCC=C(C)C]

In an embodiment, R⁷ is C₃₋₇cycloalkyl which is unsubstituted or substituted with one to three substituents independently selected from OH, NH₂, F, Cl, CN, NO₂, COOH, NHC₁₋₆alkyl, N(C₁₋₄alkyl)(C₁₋₄alkyl), C₁₋₄alkyl and OC₁₋₄alkyl. In an embodiment, the C₃₋₇cycloalkyl in R⁷ is cyclopropyl, cyclobutyl, cyclopentyl, cyclohexyl or cycloheptyl. In an embodiment, the C₃₋₇cycloalkyl in R⁷ is cyclohexyl.

In an embodiment, R⁷ is aryl or C₅₋₁₀heteroaryl which are unsubstituted or substituted with one or more substituents independently selected from OH, NH₂, F, Cl, CN, NO₂, COOH, NHC₁₋₆alkyl, N(C₁₋₄alkyl)(C₁₋₄alkyl), C₁₋₄alkyl and OC₁₋₄alkyl the latter 4 groups being unsubstituted or further substituted with one or more substituents independently selected from OH, halo, C₁₋₄alkyl, C₁₋₄fluoroalkyl, C₂₋₄alkenyl, OC₁₋₄alkyl and OC₁₋₆fluoroalkyl. In an embodiment, R⁷ is aryl or C₅₋₆heteroaryl which are unsubstituted or substituted with one to three substituents independently selected from OH, NH₂, F, Cl, CN, NO₂, COOH, NHC₁₋₆alkyl, N(C₁₋₄alkyl)(C₁₋₄alkyl), C₁₋₄alkyl, and OC₁₋₄alkyl the latter 4 groups being unsubstituted or further substituted with one or more halo. In an embodiment, R⁷ is aryl which is unsubstituted or substituted with one to three substituents independently selected from OH, NH₂, F, Cl, CN, NO₂, COOH, NHC₁₋₆alkyl, N(C₁₋₄alkyl)(C₁₋₄alkyl), C₁₋₄alkyl, and OC₁₄alkyl the latter 4 groups being unsubstituted or further substituted with one or more halo. In an embodiment, R⁷ is aryl which is unsubstituted or substituted with one to three substituents independently selected from OH, NH₂, F, Cl, CN, NO₂, COOH, C₁₋₄alkyl, and OC₁₋₄alkyl the latter 2 groups being unsubstituted or further substituted with one or more halo. In an embodiment, R⁷ is aryl which is unsubstituted or substituted with one to three substituents independently selected from OH, NH₂, F, Cl, CN, NO₂, COOH, C₁₋₄alkyl, C₁₋₄haloalkyl, OC₁₋₄alkyl, and OC₁₋₄haloalkyl. In an embodiment, R⁷ is aryl which is unsubstituted or substituted with one to three substituents independently selected from OH, NH₂, F, Cl, CN, NO₂, COOH, C₁₋₄alkyl, C₁₋₄fluoroalkyl, OC₁₋₄alkyl, and OC₁₋₄fluorolkyl. In an embodiment, R⁷ is phenyl which is unsubstituted or substituted with one to three substituents independently selected from OH, NH₂, F, Cl, CN, NO₂, COOH, C₁₋₄alkyl, C₁₋₄fluoroalkyl, OC₁₋₄alkyl, and OC₁₋₄fluorolkyl.

In an embodiment, R⁸ is selected from H, C₁₋₄alkyl and fluoroC₁₋₄alkyl. In an embodiment, R⁸ is selected from H, CH₃ and CF₃. In an embodiment, R⁸ is selected from H and CH₃.

In an embodiment, the compound of Formula (III) is selected from

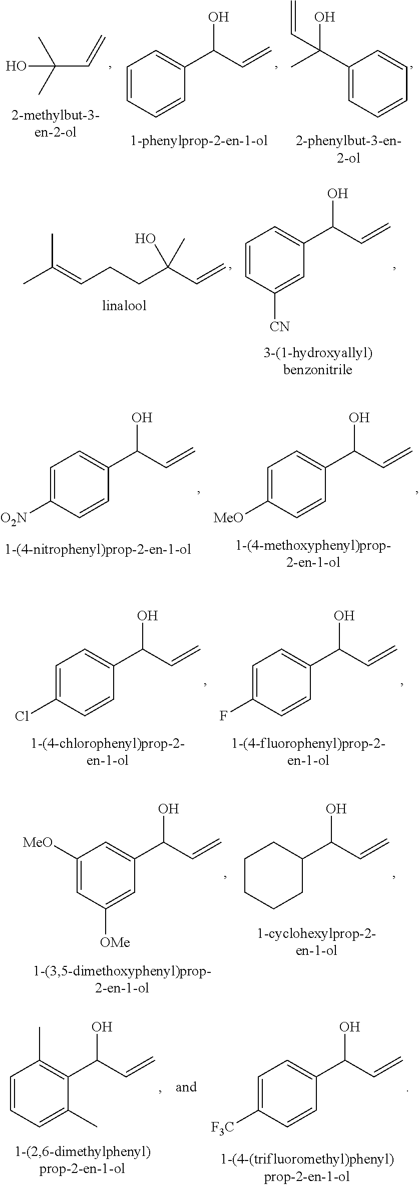

In an embodiment, the compound of Formula (III) is selected from

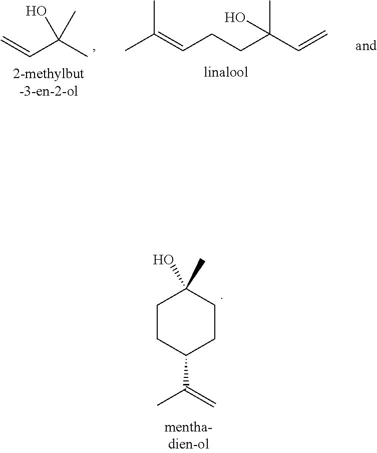

In an embodiment, the compound of Formula (III) is selected from

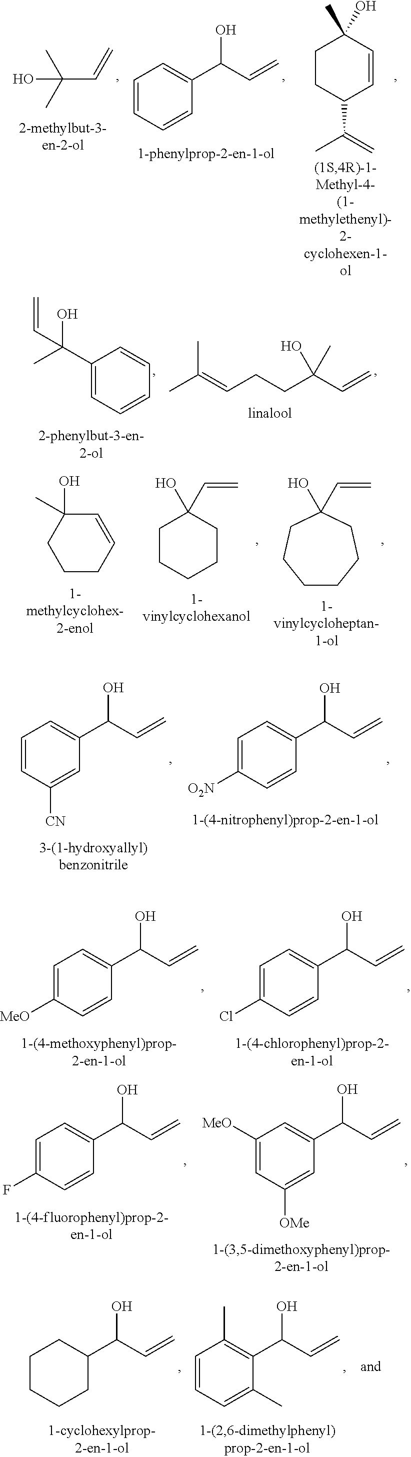

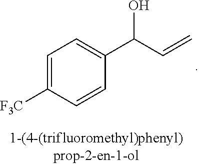

In an embodiment, the compound of Formula (I) comprises a prenyl functional group

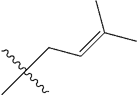 [SMILES: *CC=C(C)C]

or repeating prenyl functional groups, and the compound of Formula (I) is selected from a prenylated or polyprenylated cannabinoid, phenol, resorcinol, chalconoid, moracin, stilbenoid, polycyclic aromatic, flavanonol, isoflavanonol, flavonol, isoflavonol, chromone, coumarin and xanthone.

In an embodiment, the compound of Formula (I) is selected from a cannabinoid. In an embodiment, the cannabinoid is selected from cannabidiol, cannabidivarin, cannabigerol, cannabigerorcin and cannabigerivarin. In an embodiment, the compound of Formula (I) is cannabidiol. In an embodiment, the compound of Formula (I) is selected from, cannabidivarin, cannabigerol, grifolin, cannabigerorcin, piperogalin and cannabigerivarin. In an embodiment, the compound of Formula (I) is selected from cannabigerol, piperogalin and grifolin.

In R⁵ and R⁷ in the compound of Formula (III) are linked together to form isopiperitenol and the compound of Formula (I) is cannabidiol.

In an embodiment, the compound of Formula (I) is a natural compound. In an embodiment, the compound of Formula (I) is a naturally occurring phenolic compound. In an embodiment, the compound of Formula (I) is selected from a class of compound comprising cannabinoids, phenols, resorcinols, chalconoids, moracins, stilbenoidd, polycyclic aromatics, flavanonols, isoflavanonols, flavonols, isoflavonols, chromones, coumarins and xanthones.

It would be appreciated by a person skilled in the art R¹, R², R³ and R⁴ in a compound of Formula (II) and R⁵, R⁶, R⁷ and R⁸ in a compound of Formula (II) would correspond to R¹, R², R³, R⁴, R⁵, R⁶, R⁷ and R⁸ selected for the compound of Formula (I).

It would also be appreciated by a person skilled in the art that the compounds of Formula (I) can be further reacted to form further scaffolds of interest. For example, exemplary compounds of Formula (I), cannabidiol and cannabidivarin, can be further cyclized to form tetrahydrocannabinol and tetrahydrocannabivarin, respectively. Conditions for cyclization would be known a person skilled in the art.

In an embodiment, the compound of Formula (I) is selected from the compounds listed below:

| Compound I.D. | Structure |
|---|---|
| I-1 | 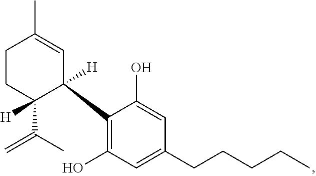 [SMILES: C=C(C)[C@@H]1CCC(C)=C[C@H]1c1c(O)cc(CCCCC)cc1O] |
| I-2 | 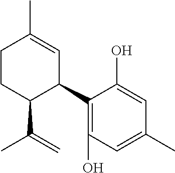 [SMILES: C=C(C)[C@H]1CCC(C)=C[C@H]1c1c(O)cc(C)cc1O] |
| I-3 | 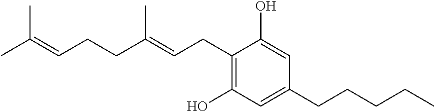 [SMILES: CCCCCc1cc(O)c(C/C=C(\C)CCC=C(C)C)c(O)c1] |
| I-4 | 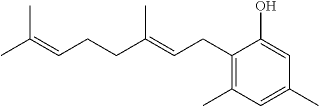 [SMILES: CC(C)=CCC/C(C)=C/Cc1c(C)cc(C)cc1O] |
| I-5 | 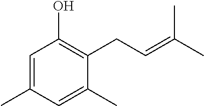 |
| I-6 | 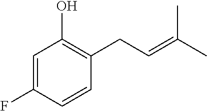 |
| I-7 | 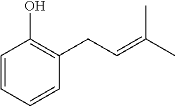 |
| I-8 | 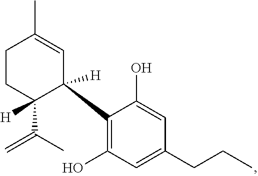 [SMILES: C=C(C)[C@@H]1CCC(C)=C[C@H]1c1c(O)cc(CCC)cc1O] |
| I-9 | 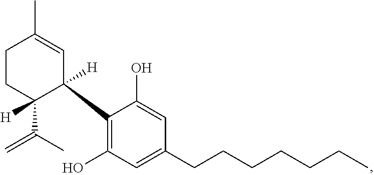 [SMILES: C=C(C)[C@@H]1CCC(C)=C[C@H]1c1c(O)cc(CCCCCCC)cc1O] |
| I-10 | 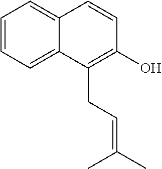 [SMILES: CC(C)=CCc1c(O)ccc2ccccc12] |
| I-11 | 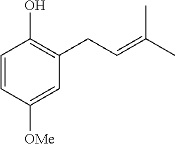 |
| I-12 | 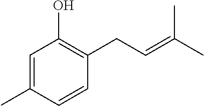 |
| I-13 | 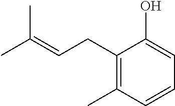 |
| I-14 | 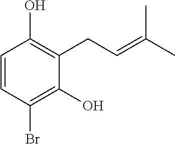 |
| I-15 | 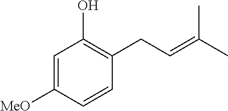 |
| I-16 | 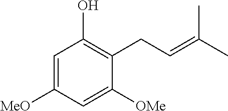 [SMILES: COc1cc(O)c(CC=C(C)C)c(OC)c1] |
| I-17 | 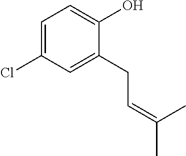 |
| I-18 | 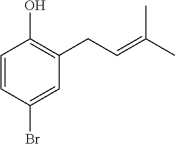 |
| I-19 | 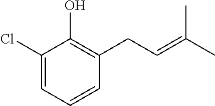 |
| I-20 | 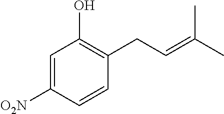 |
| I-21 | 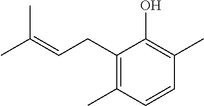 |
| I-22 | 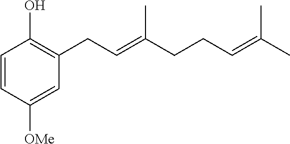 [SMILES: COc1ccc(O)c(C/C=C(\C)CCC=C(C)C)c1] |
| I-23 | 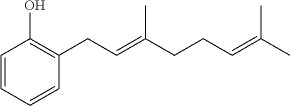 [SMILES: CC(C)=CCC/C(C)=C/Cc1ccccc1O] |
| I-24 | 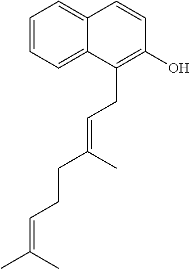 [SMILES: CC(C)=CCC/C(C)=C/Cc1c(O)ccc2ccccc12] |
| I-25 | 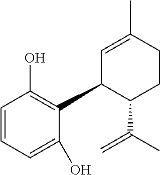 [SMILES: C=C(C)[C@H]1CCC(C)=C[C@@H]1c1c(O)cccc1O] |
| I-26 | 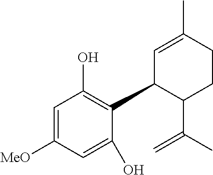 [SMILES: C=C(C)C1CCC(C)=C[C@@H]1c1c(O)cc(OC)cc1O] |
| I-27 | 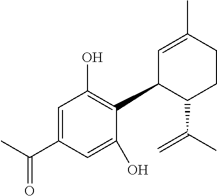 |
| I-28 | 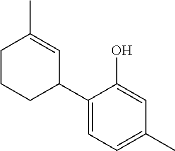 |
| I-29 | 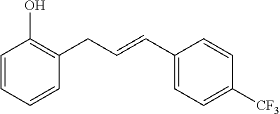 [SMILES: Oc1ccccc1C/C=C/c1ccc(C(F)(F)F)cc1] |
| I-30 | 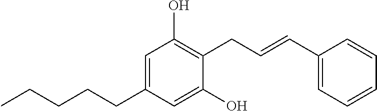 [SMILES: CCCCCc1cc(O)c(C/C=C/c2ccccc2)c(O)c1] |
| I-31 | 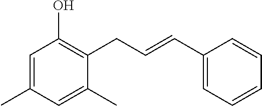 [SMILES: Cc1cc(C)c(C/C=C/c2ccccc2)c(O)c1] |
| I-32 | 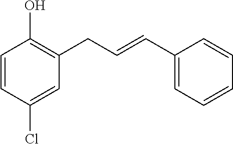 [SMILES: Oc1ccc(Cl)cc1C/C=C/c1ccccc1] |
| I-33 | 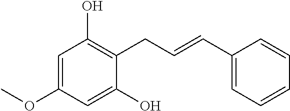 [SMILES: COc1cc(O)c(C/C=C/c2ccccc2)c(O)c1] |
| I-34 | 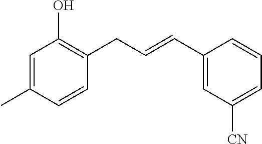 [SMILES: Cc1ccc(C/C=C/c2cccc(C#N)c2)c(O)c1] |
| I-35 | 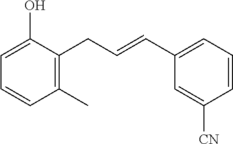 [SMILES: Cc1cccc(O)c1C/C=C/c1cccc(C#N)c1] |
| I-36 | 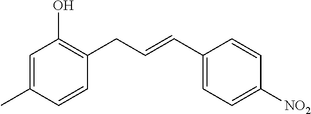 [SMILES: Cc1ccc(C/C=C/c2ccc([N+](=O)[O-])cc2)c(O)c1] |
| I-37 | 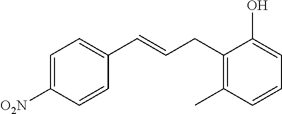 [SMILES: Cc1cccc(O)c1C/C=C/c1ccc([N+](=O)[O-])cc1] |
| I-38 | 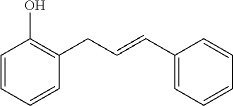 [SMILES: Oc1ccccc1C/C=C/c1ccccc1] |
| I-39 | 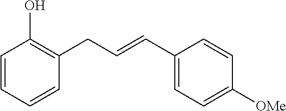 [SMILES: COc1ccc(/C=C/Cc2ccccc2O)cc1] |
| I-40 | 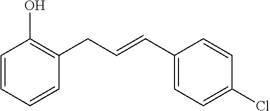 [SMILES: Oc1ccccc1C/C=C/c1ccc(Cl)cc1] |
| I-41 | 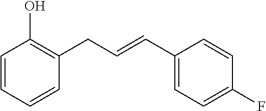 [SMILES: Oc1ccccc1C/C=C/c1ccc(F)cc1] |
| I-42 | 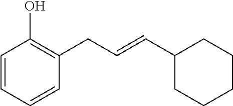 [SMILES: Oc1ccccc1C/C=C/C1CCCCC1] |
| I-43 | 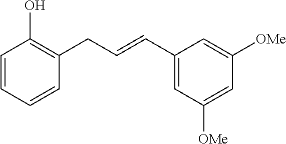 [SMILES: COc1cc(/C=C/Cc2ccccc2O)cc(OC)c1] |
| I-44 | 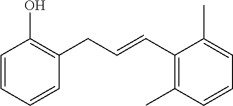 [SMILES: Cc1cccc(C)c1/C=C/Cc1ccccc1O] |
| I-45 | 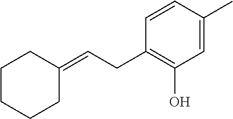 [SMILES: Cc1ccc(CC=C2CCCCC2)c(O)c1] |
| I-46 | 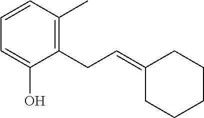 [SMILES: Cc1cccc(O)c1CC=C1CCCCC1] |
| I-47 | 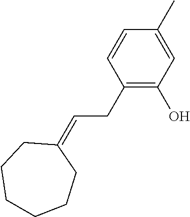 [SMILES: Cc1ccc(CC=C2CCCCCC2)c(O)c1] |
| I-48 | 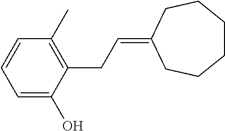 [SMILES: Cc1cccc(O)c1CC=C1CCCCCC1] |
| I-49 | 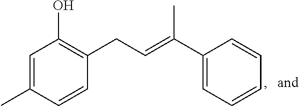 [SMILES: C/C(=C\Cc1ccc(C)cc1O)c1ccccc1] |
| I-50 | 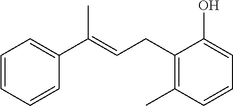 [SMILES: C/C(=C\Cc1c(C)cccc1O)c1ccccc1] |

In an embodiment, the present application includes a process for preparing a compound of Formula (I-A):

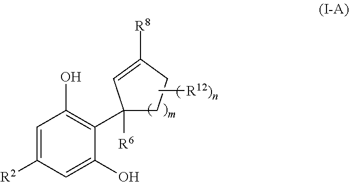

comprising reacting a compound of Formula (11):

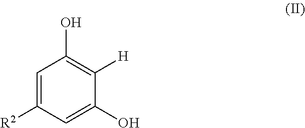

with a compound of Formula (III-A):

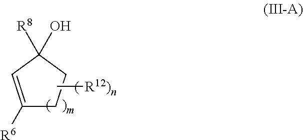

in presence of aluminum compound selected from alumina and aluminum alkoxides and in a non-protic solvent to form the compound of Formula (I-A),
wherein:
R² is selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl;
Z is selected from O, C(O), C₀₂, S, SO₂, SO, and NR¹⁰;
R⁶ is selected from H, C₁₋₆alkyl and fluoroC₁₋₆alkyl;
R^{a} is selected from H, C₁₋₆alkyl and fluoroC₁₋₆alkyl;
R¹⁰ is selected from C₁₋₆alkyl and C₁₋₆fluoroalkyl;
each R¹² is independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl;
m is 1, 2, 3, 4, 5, 6, 7, or 8; and
n is 1, 2, 3 or 4.

In an embodiment, the compound of Formula (II) is

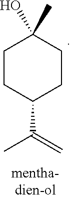

A person skilled in the art would appreciate that when R¹ is H, compounds of Formula I can further react with compounds Formula III to form di-ortho-allylated hydroxy phenyl compounds. Therefore, in an embodiment, the process of the application provides di-ortho-allylated hydroxy phenyl compounds of Formula (I-B).

Accordingly, in an embodiment, when R¹ is H, the application includes a process for preparing a compound of Formula I-B

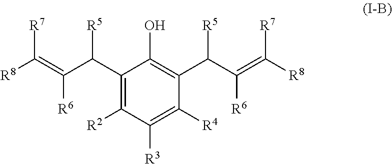

comprising reacting a compound of Formula (II):

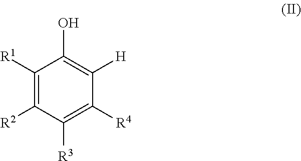

with a compound of Formula (III):

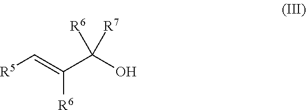

in presence of aluminum compound selected from alumina and aluminum alkoxides and in a non-protic solvent to form the compound of Formula (I),
wherein R¹, R², R³, R⁴, R⁵, R⁶, R⁷, R⁸ are as defined above for the compounds of Formula I, II and II.

In an embodiment, the compound of Formula (I-B) is selected from the compounds listed below:

| Compound I.D. | Structure |
|---|---|
| I-51 | 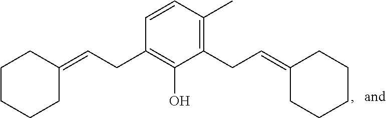 [SMILES: Cc1ccc(CC=C2CCCCC2)c(O)c1CC=C1CCCCC1] |
| I-52 | 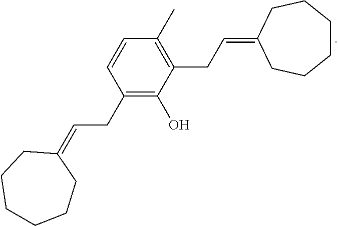 [SMILES: Cc1ccc(CC=C2CCCCCC2)c(O)c1CC=C1CCCCCC1] |

In an embodiment, the aluminum compound is alumina. In an embodiment, the alumina is neutral, basic or acidic alumina. In an embodiment, the alumina is neutral alumina. In an embodiment, the alumina is basic alumina. In an embodiment, the alumina is acidic alumina. In an embodiment, the alumina (e.g., neutral, basic and acidic alumina) is available from commercial sources.

In an embodiment, the alumina is basic alumina. In an embodiment, the basic alumina, has a pH of greater than about 7.5, about 8, about 8.5, about 9.0, about 9.5, about 10 or about 10.5. In an embodiment, the basic alumina has a pH of greater than about 9.0, about 9.5, about 10 or about 10.5. In an embodiment, the basic alumina has a pH of about 10.

In an embodiment, the alumina is neutral alumina. In an embodiment, the neutral alumina has a pH of about 7.

In an embodiment, the alumina is acidic alumina. It would be appreciated by a person skilled in the art that acid can be added to the alumina in process of the application. In an embodiment the acid is selected from a Lewis acid and a Bronsted acid, and a combination thereof. In an embodiment, the Lewis acid is selected from boron trichloride, boron trifluoride, boron trifluoride diethyl etherate, iron (Ill) bromide, iron (Ill) chloride, aluminum chloride, aluminum bromide, tin (IV) chloride, titanium (IV) chloride, and titanium (IV) isopropoxide and a combination thereof. In an embodiment, the Bronsted acid is selected from hydrochloric acid, sulfuric acid, nitric acid, phosphoric acid, acetic acid, trifluoroacetic acid, toluene sulfonic acid, trichloroacetic acid, boric acid, oleic acid, palmitic acid, and camphor sulfonic acid and a combination thereof.

In an embodiment, the aluminum compound is an aluminum alkoxide. In an embodiment, the aluminum alkoxide is an aluminum C₁₋₁₀alkoxide. In an embodiment, the aluminum alkoxide is an aluminum C₁₋₆alkoxide. In an embodiment, the aluminum alkoxide is an aluminum C₁₋₆alkoxide. In an embodiment, the aluminum alkoxide is selected from aluminum methoxide, aluminum ethoxide, aluminum-n-propoxide, aluminum isopropoxide, aluminum-n-butoxide, aluminum-sec-butoxide, aluminum-iso-propoxide and aluminum tert-butoxide. In an embodiment, the aluminum alkoxide is aluminum isopropoxide. In an embodiment, the aluminum alkoxide (e.g aluminum isopropoxide) is available from commercial sources.

In an embodiment, the aluminum alkoxide dissolves in the non-protic solvent. Therefore, under these conditions the reaction of the compound of Formula (II) with the compound of Formula (III) or (III-A) is a homogenous reaction.

In an embodiment, the non-protic solvent is a mixture of one or more non-protic solvents. In an embodiment, the non-protic solvent, suitably non-protic organic solvent, is a non-polar solvent or a polar aprotic solvent. In an embodiment, the non-polar solvent comprises hydrophobic solvents. In an embodiment, the non-protic solvent is selected from hexane, hexanes, heptane, heptanes, cyclohexane, petroleum ether, octane, diglyme, toluene, xylenes, benzene, chloroform, fluorinated alkanes, dichloromethane (DCM), 1,2-dichloroethane (DCE), ethyl acetate, carbon tetrachloride, tetrahydrofuran (THF), diethyl ether, diisopropyl ether, isooctane, methyl ethyl ketone, acetone, dimethyl sulfoxide, dimethylformamide, methyl tert-butyl ether, trichloroethane, n-butyl acetate, chlorobenzene acetonitrile, and trifluorotoluene, and mixtures thereof. In an embodiment, the non-protic solvent is selected from hexane, hexanes, heptane, heptanes, cyclohexane, petroleum ether, octane, diglyme, toluene, xylenes, benzene, chloroform, fluorinated alkanes, dichloromethane (DCM), 1,2-dichloroethane (DCE), ethyl acetate, carbon tetrachloride, tetrahydrofuran (THF), diethyl ether, diisopropyl ether, isooctane, methyl ethyl ketone, methyl tert-butyl ether, trichloroethane, n-butyl acetate, chlorobenzene acetonitrile, and trifluorotoluene, and mixtures thereof. In an embodiment, the non-protic solvent is a hydrophobic solvent selected from hexane, hexanes, heptane, heptanes, cyclohexane, toluene, xylene, dichloromethane and 1,2-dichloroethane. In an embodiment, the hydrophobic solvent is selected from hexane, hexanes, toluene, dichloromethane and 1,2-dichloroethane. In an embodiment, the hydrophobic solvent is hexanes. In an embodiment, the hydrophobic solvent is 1,2-dichloroethane.

The Applicants have shown that the compound of Formula (I) and (I-A) can be formed by reacting the compound of Formula (II) with a compound of Formula (III) or (III-A) in the presence of alumina and further additives including dehydrating reagents such as and magnesium sulfate and/or various acids. Therefore, in an embodiment, the process of the application comprises reacting the compound of Formula (II) with a compound of Formula (III) or (III-A) in the presence of alumina and a dehydrating agent and in a non-protic solvent to form the compound of Formula (I) or (I-A). In an embodiment, the dehydrating agent is selected from magnesium sulfate, sodium sulfate, aluminum phosphate, calcium oxide, cyanuric chloride, orthoformic acid, phosphorus pentoxide, sulfuric acid and molecular sieves, and combinations thereof. In an embodiment, the dehydrating agent is selected from magnesium sulfate, sodium sulfate, aluminum phosphate, calcium oxide, cyanuric chloride, orthoformic acid, phosphorus pentoxide, and molecular sieves, and combinations thereof. In an embodiment, the dehydrating agent is magnesium sulfate.

In an embodiment, the forming of the compounds of Formula (I) or (I-A) comprises mixing the compounds of Formula (II), the compounds of Formula (III) or (III-A) and the aluminum compound, and any optional additives, in the non-protic solvent under continuous flow reaction conditions using for example continuous processors. Continuous flow processors comprise a combination of mixing and conveying means that allow the reactants to flow into or through a mixing means, react to form products and allow the products to flow out of the mixing means for isolation and purification on a continuous basis. In the mixing and conveying means, the reaction conditions (such as temperature and pressure) can be controlled. Such continuous flow processors are well known in the art. In an embodiment, the flow reaction conditions comprise a heterogeneous reactor comprising for example a fixed bed reactor, a trickle bed reactor, a moving bed reactor or a rotation bed reactor. In an embodiment, the aluminum compound is comprised in the bed reactor and the other reagents, including the compounds of Formula (II) and (Ill) or (III-A) and optional additives flow through the bed to be converted into compounds of Formula (I) or (I-A).

In an embodiment, the forming of the compounds of Formula (I) or (I-A) comprises mixing the compounds of Formula (II), the compounds of Formula (III) or (III-A) and the aluminum compound, and any optional additives, in the non-protic solvent under batch reaction conditions.

In an embodiment, when forming the compound of Formula (I) or (I-A), the process further comprises mixing the compound of Formula (II), the compound of Formula (III) or (III-A) and the aluminum compound, and any optional additives, in the non-protic solvent with the addition of excess amounts of the compound of Formula (II). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 1.1 to about 5, about 1.1 to about 4, about 1.1 to about 3, about 2 to about 5, about 2 to about 4, about 3 to about 4, or about 1.5 to about 3 molar equivalents of the compound of Formula (II) relative to the amount of the compound of Formula (III) or (III-A). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 1 to about 5, about 1 to about 4, about 1 to about 3, or about 1.5 to about 3 molar equivalents of the compound of Formula (II) relative to the amount of the compound of Formula (III) or (III-A). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 1 to about 5, about 1 to about 4, about 1 to about 3, or about 1.5 to about 3, about 2 to about 5, about 2 to about 4, about 2.5 to about 3.5, or about 3 molar equivalents of the compound of Formula (II) relative to the amount of the compound of Formula (III) or (III-A). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 2 to about 4, about 2.5 to about 3.5, or about about 3 molar equivalents of the compound of Formula (II) relative to the amount of the compound of Formula (III) or (III-A). In an embodiment, the forming of the compound of Formula (I) comprises mixing the compound of Formula (II), the compound of Formula (III) or (III-A) and the aluminum compound, and any optional additives, in the non-protic solvent with the addition of, for example, about 1.5 molar equivalents of the compound of Formula (II) relative to the amount of the compound of Formula (III) or (III-A). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 1.1, about 1.5, about 2, about 2.5, about 3, about 3.5, or about 4 molar equivalents of the compound of Formula (II) relative to the amount of the compound of Formula (III) or (III-A). In an embodiment, the forming of the compound of Formula (I) or (I-A) comprises mixing the compound of Formula (II), the compound of Formula (III) or (III-A) and the aluminum compound, and any optional additives, in the non-protic solvent with the addition of, for example, about 3 molar equivalents of the compound of Formula (II) relative to the amount of the compound of Formula (III) or (III-A).

In an embodiment, when it is desired to add additional allyl groups, beyond the ortho-allyl group (i.e. a polyallylated hydroxy aryl compound such as a di-, tri- and tetra-allylated hydroxy aryl compound), the forming of the polyallylated hydroxy aryl compound further comprises mixing the compound of Formula (II), the compound of Formula (III) or (III-A) and the aluminum compound, and any optional additives, in the non-protic solvent with the addition of excess amounts of the compound of Formula (III) or (III-A). In an embodiment, the forming of the polyallylated hydroxy aryl compound comprises mixing the compound of Formula (II), the compound of Formula (III) or (III-A) and aluminum compound, and any optional additives, in the non-protic solvent with the addition of, for example, about 1.1 to about 5, about 1.1 to about 4, about 1.1 to about 3, about 2 to about 5, 1.5 to about 4, about 2 to about 4, about 3 to about 4, about 3 to about 5, about 4 to about 5, or about 1.5 to about 3 molar equivalents of the compound of Formula (III) or (III-A) relative to the amount of the compound of Formula (II).

In an embodiment, the aluminum compound present in an amount of about 1 g to about 3 g, about 1.5 g to about 3 g, or about 1.5 g to about 2 g per 1 mmol of the compound of Formula (III) or (III-A). In an embodiment, the aluminum compound is present in an amount of about 2 g per 1 mmol of the allylic alcohol.

In an embodiment, the forming of the compound of Formula (I) or (I-A) further comprises mixing the compound of Formula (II), the compound of Formula (III) or (III-A) and the aluminum compound, and any optional additives, in the non-protic solvent to form a reaction mixture and heating the reaction mixture. In an embodiment the reaction mixture is heated to the boiling point (refluxing temperature) of the solvent. In an embodiment, the reaction mixture is heated to about 40° C. to about 83° C., about 60° C. to about 83° C., about 70° C. to about 83° C., or about 83° C. In an embodiment, the reaction mixture is heated to about 40° C. to about 85° C., about 60° C. to about 85° C., about 70° C. to about 85° C., or about 85° C. In an embodiment, the reaction mixture is heated for about 4 hours to about 24 hours, about 6 hours to about 24 hours, or about 12 hours to 24 hours. In an embodiment, the reaction mixture is heated at refluxing temperature of the solvent for about 24 hours.

In an embodiment, the reaction mixture is heated under microwave synthesis conditions. In an embodiment, the microwave synthesis conditions comprise heating the reaction mixture in a microwave reactor. In an embodiment, the microwave synthesis conditions comprise heating the reaction mixture in a microwave reactor to about 100° C. to about 175° C., about 125° C. to about 175° C., or about 150° C.

In an embodiment, after heating, the reaction mixture is cooled and filtered through a filter agent, such as Celite® or silica, and the filtrate is concentrated for example, by evaporation such as rotoevaporation, to provide a crude product that comprises the compound of Formula (I) or (I-A). In an embodiment, the crude product is then purified using chromatography such as column chromatography using a suitable solvent or mixture of solvents, or any other known purification method. In an embodiment, the column chromatography is flash column chromatography.

In an embodiment, the crude product is purified by crystallization. In an embodiment, the crude product is purified by crystallization without the use of chromatography. In an embodiment, the crude product is crystallized using hexane, hexanes, heptane, heptanes, cyclohexane, toluene, xylene and the like. In an embodiment, the crude product is a crude ortho-allylated cannabinoid and the crude product is crystallized using hexane, hexanes, heptane, heptanes, or cyclohexane. In an embodiment, the crude product is a crude ortho-allylated cannabinoid and the crude product is crystallized with heptane.

In an embodiment, the crude product is purified by distillation. In an embodiment, the crude product is purified by distillation without the use of chromatography. In an embodiment, the crude product is a crude ortho-allylated cannabinoid and the crude product is purified by distillation.

In an embodiment, the process of the application can be performed consecutively such that the ortho-allylated hydroxy phenyl compound formed from a first process of the application is used as the hydroxy phenyl compound in a subsequent process of the application. Accordingly, in the embodiment, the hydroxy phenyl compound is the ortho-allylated hydroxy phenyl formed by a process of the application described above.

In an embodiment, the process provides the compound of Formula (I) or (I-A) as the major product of the process. In an embodiment, the process provides the compound of Formula (I) or (I-A) in a yield of greater than about 15%, about 20%, about 25%, about 30%, about 35%, about 40%, about 45%, about 50%, about 55%, about 60%, about 65%, about 70%, about 75%, about 80%, about 85%, about 90% or about 95%. In an embodiment, the process provides the compound of Formula (I) or (I-A) in a yield of greater an about 55%, about 60%, about 65%, about 70%, about 75%, about 80%, about 85%, about 90% or about 95%. In an embodiment, the process provides the compound of Formula (I) or (I-A) in a yield of greater an about 70%, about 75%, about 80%, about 85%, about 90% or about 95%.

In an embodiment, the process selectively forms the compound of Formula (I) or (I-A) as the major isomer. Accordingly, in an embodiment, the present application also includes a process for selectively preparing a compound of Formula (I) or (I-A) comprising reacting a compound of Formula (II) with a compound of Formula (III) or (III-A) in presence of an aluminum compound and in a non-protic solvent to form the compound of Formula (I) or (I-A), wherein the compounds of Formulae (I), (I-A), (II), (Ill) and (III-A) are as defined above.

A person skilled in the art would appreciate that further manipulation of the substituent groups using known chemistry can be performed on the intermediates and final compounds in the Schemes above to provide alternative compounds of the application.

Salts of compounds of the application may be formed by methods known to those of ordinary skill in the art, for example, by reacting a compound of the application with an amount of acid or base, such as an equivalent amount, in a medium such as one in which the salt precipitates or in aqueous medium followed by lyophilization.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### EXAMPLES

The following non-limiting examples are illustrative of the present application.

### A: Synthesis of Exemplary Compounds of Formula I

### General Experimental Procedures.

Alumina was purchased from Millipore Sigma (activated, acidic, Brockmann I, catalogue #199966; activated neutral, Brockmann I, catalogue #199974; activated, basic, Brockmann I, catalogue #199443). Substrates were purchased from AKScientific and used as obtained. Solvents were purchased from Fisher Scientific, reagent grade, and used without further purification.

¹H NMR spectra were acquired at 700 MHz with a default digital resolution (Bruker parameter: FIDRES) of 0.15 Hz/point. Coupling constants reported herein therefore have uncertainties of ±0.30 Hz. Chemical shifts in ¹H NMR and ¹³C NMR spectra are reported in parts per million (ppm) with reference to residual chloroform (δ_{H} 7.26) and deuterated chloroform (δ_{C} 77.16). Peak multiplicities are reported using the following abbreviations: s, singlet; d, doublet; t, triplet; q, quartet; dd, doublet of doublets; m, multiplet. Reaction progress was monitored by thin layer chromatography (TLC, EMD Chemicals, Inc., silica gel 60 F254). TLC plates were developed via capillary action in hexane-ethyl acetate solvent mixtures then visualized under UV light followed by p-anisaldehyde stain. An automated flash chromatography system (Teledyne CombiFlash R_{f} 200) was used for the purification of compounds on silica gel (either 40-60 μM particle size).

### Example 1: Synthesis of Cannabidiol (I-1)

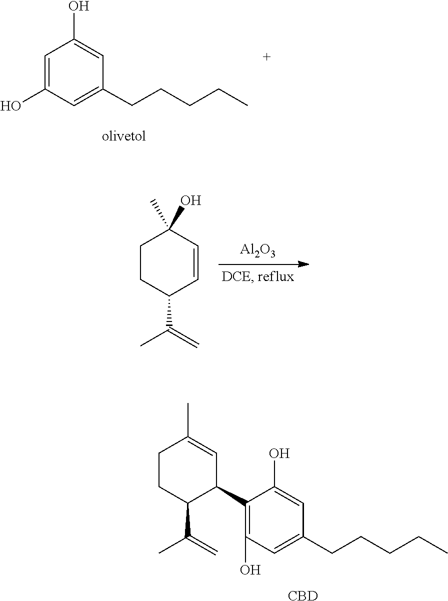

To a round bottom flask were added olivetol (270 mg, 1.5 mmol), (1R,4R)-1-methyl-4-(prop-1-en-2-yl)cyclohex-2-en-1-ol (76 mg, 0.5 mmol), acidic alumina (1.0 g) and dichloroethane (5 mL). The reaction mixture was stirred at reflux temperature (82° C.) for 3 h. The reaction was filtered through a fritted funnel and solids were washed with dichloroethane. The solvent was removed, and the remaining residue was purified by flash column chromatography on silica gel using gradient elution with ethyl acetate and hexanes to obtain cannabidiol (CBD) as an oil (61 mg, 0.19 mmol, 39% yield).

¹H NMR (400 MHz, CDCl₃) δ 6.22 (app. q, J=2.7 Hz, 2H), 6.09 (s, 1H), 5.53 (s, 1H), 5.01 (s, 1H), 4.66-4.64 (m, 1H), 4.47 (s, 1H), 3.56-3.51 (m, 1H), 2.63-2.55 (m, 1H), 2.51-2.45 (m, 1H), 2.30-2.18 (m, 2H), 2.13-2.07 (m, 1H), 1.89-1.72 (m, 5H), 1.54 (s, 3H), 1.51-1.43 (m, 1H), 1.37-1.25 (m, 4H), 0.89 (t, J=7.1 Hz, 3H).

### Synthesis of 4,5′-dimethyl-2′-(prop-1-en-2-yl)-1′,2′,3′,4′-tetrahydro-[1,1′-biphenyl]-2,6-diol (I-2)

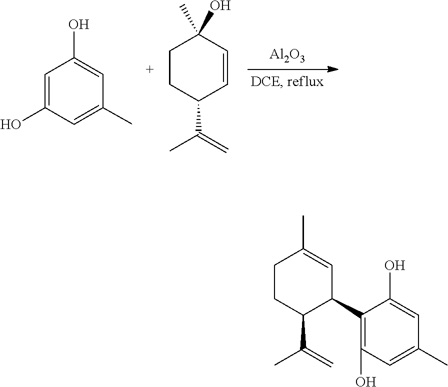

To a round bottom flask containing DCE (10 mL) was added acidic alumina (0.66 g; 2 g/mmol allyl alcohol), 3,5-dihydroxytoluene (0.12 g, 0.99 mmol, 3 equiv.) was added followed by cis-Isolimonenol (0.05 ml, 0.33 mmol, 1.0 equiv.) and the reaction was heated to 85° C. for three hours. The reaction mixture was cooled to room temperature and filtered through a fritted funnel eluting with 100 ml of DCE. The solution was concentrated and purified by column chromatography using an ethylacetate/hexanes gradient of 5-10% providing the desired compound as a colourless oil (0.045 g, 0.17 mmol, 53%).

¹H NMR (400 MHz, CDCl₃) δ 6.21-6.18 (m, 2H), 6.07 (s, 1H), 5.54 (s, 1H), 4.66-4.64 (m, 1H), 4.51 (brs, 1H), 4.46 (brs, 1H), 3.58-3.52 (m, 1H), 2.45 (ddd, J=11.4, 9.8, 3.4 Hz, 1H), 2.28-2.17 (m, 2H), 2.15 (s, 3H), 2.13-2.06 (m, 1H), 1.87-1.71 (m, 5H), 1.56 (s, 3H).

### Example 2: Synthesis of CBG (I-3)

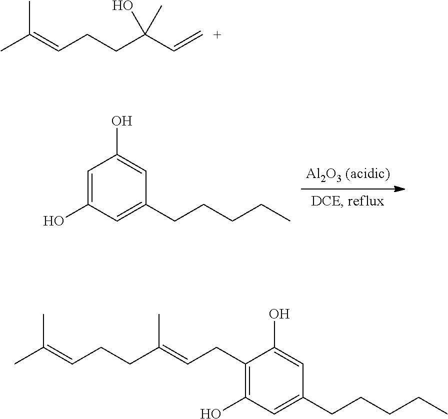

To a round bottom flask containing DCE (33 ml; dried over 4A MS) was added acidic alumina (1.3 g; 2 g/mmol linalool) under stirring. Reaction mixture was heated to 85° C. and olivetol (3.5 g, 19.44 mmol, 3 equiv.) was added followed by linalool (1.1 ml, 6.48 mmol, 1.0 equiv.). After stirring at reflux for four hours, the reaction mixture was cooled to room temperature and filtered through frit funnel eluting with 600 ml of DCE. The solution was concentrated and purified by column chromatography using an ethylacetate/hexanes gradient of 5-10% providing the desired compound as a colourless oil (0.058 g, 0.22 mmol, 15%)

¹H NMR (400 MHz, CDCl₃) δ 6.25 (s, 2H), 5.29-5.25 (m, 1H), 5.07-5.03 (m, 1H), 4.95 (brs, 2H), 3.39 (d, J=7.1 Hz, 2H), 2.47-2.44 (m, 2H), 2.11-2.04 (m, 4H), 1.81 (s, 3H), 1.68 (s, 3H), 1.59 (s, 3H), 1.57-1.53 (m, 3H), 1.35-1.26 (m, 4H), 0.88 (t, J=6.9 Hz, 3H).

### Example 3: Synthesis of CBG with Aluminum Isopropoxide (I-3)

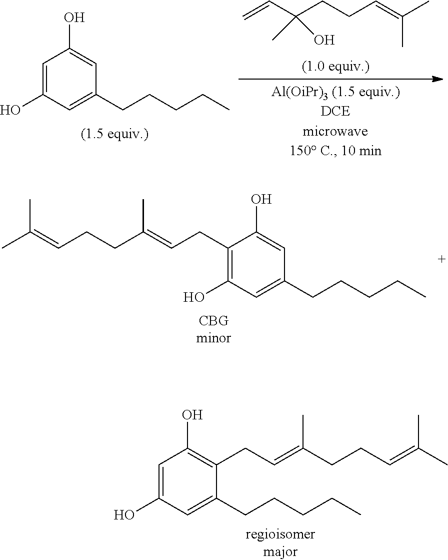 [SMILES: CCCCCc1cc(O)cc(O)c1 ; C=CC(C)(O)CCC=C(C)C ; CCCCCc1cc(O)c(C/C=C(\C)CCC=C(C)C)c(O)c1 ; CCCCCc1cc(O)cc(O)c1C/C=C(\C)CCC=C(C)C]

To linalool (72.4 mg, 0.469 mmol) in 1,2-dichloroethane (5 mL), olivetol (125.6 mg, 0.697 mmol) was added with aluminum isopropoxide (144.5 mg, 0.707 mmol). The solution was stirred in a microwave reactor at 150° C. for 10 minutes. The reaction mixture was diluted to 15 mL (1,2-dichloroethane) and washed with 2 M HCl (15 mL), followed by a brine wash (15 mL) then dried over MgSO₄ and finally concentrated in vacuo. The yields of the mixture were determined by ¹H NMR analysis of the crude reaction mixture using 1,2-dibromomethane (70.1 mg, 0.403 mmol) as an internal standard.

NMR Yield=14% CBG (0.065 mmol), 23% regioisomer (0.109 mmol) CBG:

¹H NMR (400 MHz, CDCl₃) δ 6.24 (s, 2H), 5.26 (ddq, J=7.8, 3.0, 1.3 Hz, 1H), 5.05 (m, 3H), 3.40 (d, J=7.1 Hz, 2H), 2.45 (td, J=7.8, 2.1 Hz, 2H), 2.16-2.04 (m, 4H), 1.81 (s, 3H), 1.68 (s, 3H), 1.59 (s, 3H), 1.57-1.53 (m, 2H), 1.35-1.21 (m, 4H), 0.97-0.81 (m, 3H).

LRMS (APCI) m/z: [M]⁺ Calcd for C₂₁H₃₃O₂ 317.2; Found 317.2.

### Regioisomer:

¹H NMR (400 MHz, CDCl₃) 6.26 (t, J=2.9 Hz, 1H), 6.22 (dd, J=4.2, 2.6 Hz, 1H), 5.27 (s, 1H), 5.18-5.11 (m, 1H), 5.10-5.02 (m, 1H), 4.87-4.83 (m, 1H), 3.30 (d, J=7.0 Hz, 2H), 2.60-2.48 (m, 2H), 2.11-2.00 (m, 4H), 1.80 (d, J=1.3 Hz, 2H), 1.75-1.71 (m, 2H), ), 1.68 (d, J=1.4 Hz, 2H), 1.65 (d, J=1.4 Hz, 1H), 1.59 (d, J=1.4 Hz, 2H), 1.52 (m, 2H), 1.38-1.30 (m, 4H), 0.98-0.85 (m, 3H).

LRMS (APCI) m/z: [M]⁺ Calcd for C₂₁H₃₃O₂ 317.2; Found 317.2.

### Example 4: Synthesis of (E)-2-(3,7-dimethylocta-2,6-dien-1-yl)-3,5-dimethylphenol (I-4)

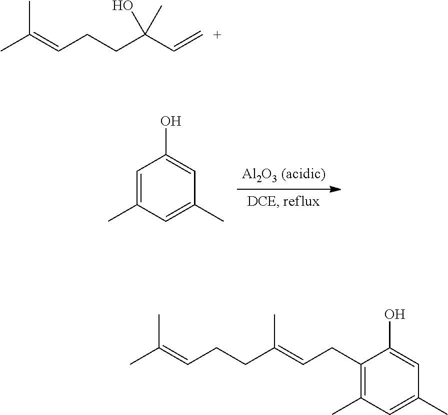

To a round bottom flask containing DCE (3 ml; dried over 4A MS) was added acidic alumina (1.3 g; 2 g/mmol linalool) under stirring. 3,5-dimethylphenol (0.24 g, 1.9 mmol, 3 equiv.) was added followed by linalool (0.11 ml, 0.65 mmol, 1.0 equiv.) and the reaction was heated to 85° C. for four hours. The reaction mixture was cooled to room temperature and filtered through frit funnel eluting with 100 ml of DCE. The solution was concentrated and purified by column chromatography using an ethylacetate/hexanes gradient of 5-10% providing the desired compound as a colourless oil (0.058 g, 0.22 mmol, 34%)

¹H NMR (400 MHz, CDCl₃) δ 6.59 (s, 1H), 6.51 (s, 1H), 5.17-5.13 (m, 1H), 5.07-5.03 (m, 1H), 4.96 (brs, 1H), 3.33 (d, J=6.8 Hz, 2H), 2.26 (s, 3H), 2.24 (s, 3H), 2.18-2.01 (m, 4H), 1.80 (s, 3H), 1.67 (s, 3H), 1.59 (s, 3H).

### Example 5: Synthesis of 3,5-dimethyl-2-(3-methylbut-2-en-1-yl)phenol (I-5)

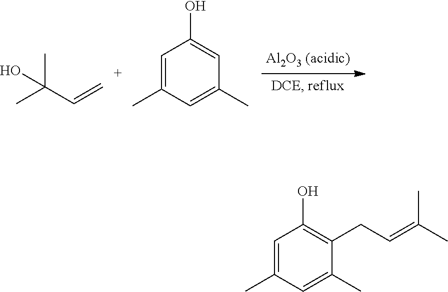

To a round bottom flask containing DCE (3 ml; dried over 4A MS) was added acidic alumina (1.2 g; 2 g/mmol allyl alcohol) under stirring. 3,5-dimethylphenol (0.21 g, 1.7 mmol, 3 equiv.) was added followed by 1,1-dimethylallyl alcohol (0.06 ml, 0.58 mmol, 1.0 equiv.) and the reaction was heated to 85° C. for five hours. The reaction mixture was cooled to room temperature and filtered through frit funnel eluting with 100 ml of DCE. The solution was concentrated and purified by column chromatography using an ethylacetate/hexanes gradient of 5-10% providing the desired compound as a colourless oil (0.047 g, 0.24 mmol, 43%).

¹H NMR (400 MHz, CDCl₃) δ 6.59 (s, 1H), 6.50 (s, 1H), 5.17-5.13 (m, 1H), 4.94 (brs, 1H), 3.33 (d, J=7.0 Hz, 2H), 2.26 (s, 3H), 2.24 (s, 3H), 1.81 (s, 3H), 1.72 (s, 3H).

### Example 6: Synthesis of 5-fluoro-2-(3-methylbut-2-en-1-yl)phenol (I-6)

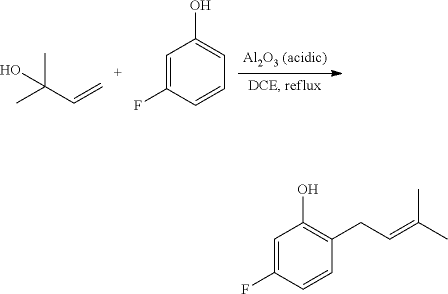

To a round bottom flask containing DCE (3 ml; dried over 4A MS) was added acidic alumina (1.2 g; 2 g/mmol allyl alcohol) under stirring. 3-fluorophenol (0.19 g, 1.7 mmol, 3 equiv.) was added followed by 1,1-dimethylallyl alcohol (0.06 ml, 0.58 mmol, 1.0 equiv.) and the reaction was heated to 85° C. for five hours. The reaction mixture was cooled to room temperature and filtered through frit funnel eluting with 100 ml of DCE. The solution was concentrated and purified by column chromatography using an ethylacetate/hexanes gradient of 5-10% providing the desired compound as a colourless oil (0.018 g, 0.1 mmol, 17%).

¹H NMR (400 MHz, CDCl₃) δ 7.05-7.00 (m, 1H), 6.59-6.53 (m, 2H), 5.40 (brs, 1H), 5.31-5.26 (m, 1H), 3.31 (d, J=7.2 Hz, 2H), 1.78 (brs, 6H).

### Example 7: Synthesis of 2-(3-methylbut-2-en-1-yl)phenol (I-7)

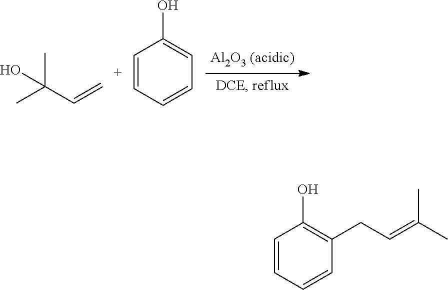

To a round bottom flask containing DCE (3 ml) was added acidic alumina (1.2 g; 2 g/mmol allyl alcohol) under stirring. Phenol (0.16 g, 1.7 mmol, 3 equiv.) was added followed by 1,1-dimethylallyl alcohol (0.06 ml, 0.58 mmol, 1.0 equiv.) and the reaction was heated to 85° C. for five hours. The reaction mixture was cooled to room temperature and filtered through frit funnel eluting with 100 ml of DCE. The solution was concentrated and purified by column chromatography using an ethylacetate/hexanes gradient of 5-10% providing the desired compound as a colourless oil (0.033 g, 0.2 mmol, 35%)

¹H NMR (400 MHz, CDCl₃) δ 7.10 (d, J=7.3 Hz, 2H), 6.86 (td, J=7.4, 1.3 Hz, 1H), 6.82-6.79 (m, 1H), 5.35-5.30 (m, 1H), 5.08 (brs, 1H), 3.36 (d, J=7.3 Hz, 2H), 1.78 (brs, 6H).

### B: Synthesis of Exemplary Compounds of Formula I Using General Procedure a

### General Procedure a

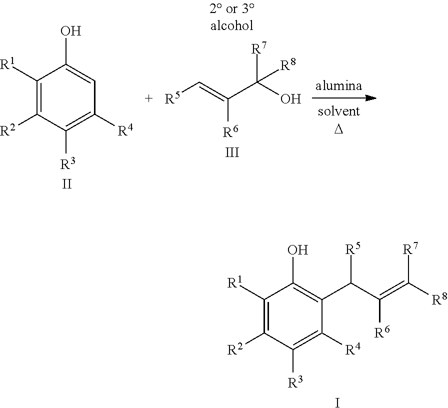

In an oven-dried round bottom flask, or sealed tube, is added allyl alcohol (III, 1 equiv.), phenol entity (II, 3 equiv.) and acidic alumina (2 g/mmol relative to allyl alcohol). The solvent of choice was added (about 0.2 M) and the flask capped or equipped with a reflux condenser. The reaction was heated in an about 85° C. oil-bath, monitored by TLC for complete consumption of the allyl alcohol. Upon reaction completion, the mixture was cooled to room temperature and the alumina filtered out. The alumina collected was rinsed with ethyl acetate until TLC indicated that product was no longer running off the alumina. The organic fractions collected were concentrated in vacuo and then purified by flash column chromatography via appropriately sized silica cartridge and eluting with a gradient of ethyl acetate:hexanes. In some embodiments, the General Procedure further provides compounds of Formula I-B.

### Exemplary Compounds of Formula III

Exemplary secondary and tertiary alcohols of Formula III are available from commercial sources, or are prepared from available precursors using were procedures known in the art. Exemplary secondary and tertiary alcohols of Formula used in the examples are shown in Scheme 1 as follows:

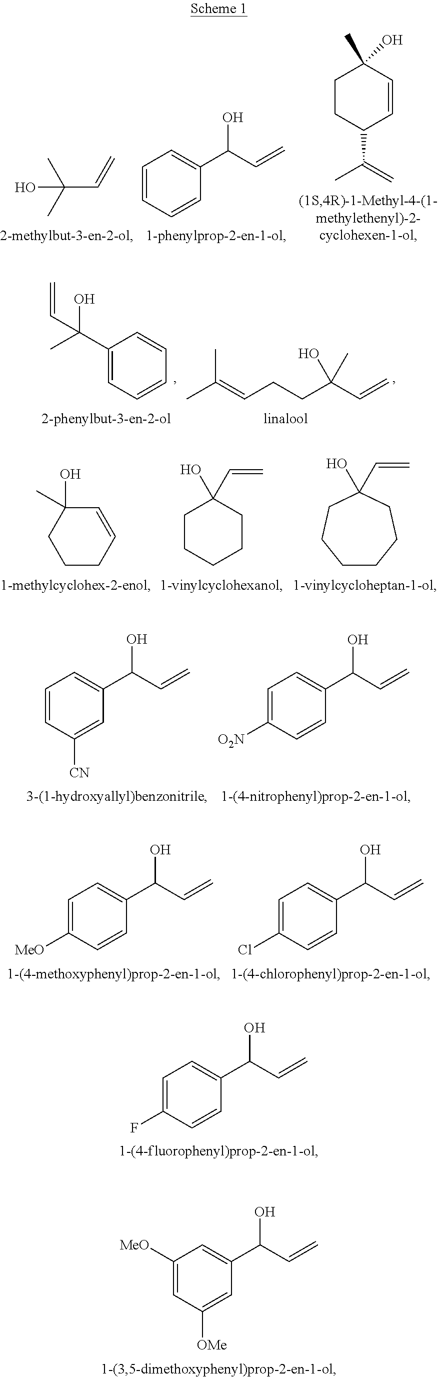

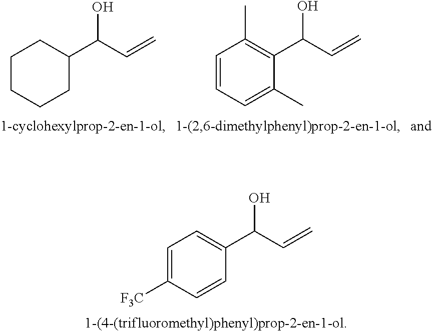

### Example 8: Synthesis of I-1

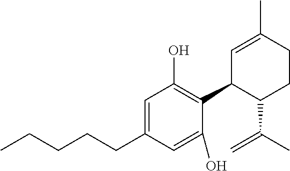

Synthesized following General Procedure A. (1S,4R)-1-Methyl-4-(1-methylethenyl)-2-cyclohexen-1-ol (0.50 g, 3.28 mmol), olivetol (1.77 g, 9.85 mmol), and alumina (6.56 g) in cyclohexane (8 mL). The reaction was complete after 2 h of heating. Product was isolated as a pale yellow oil (0.51 g, 50%) by column chromatography gradient elution 0-30% ethyl acetate:hexanes on a 40 g silica column. R_{f} (20% ethyl acetate:hexanes) 0.32. ¹H NMR (400 MHz, Chloroform-d) δ 6.20 (q, J=2.7 Hz, 2H), 6.05 (s, 1H), 5.52 (s, 1H), 4.65 (p, J=1.6 Hz, 1H), 4.56 (s, 1H), 4.46 (dd, J=1.9, 0.9 Hz, 1H), 4.46 (dd, J=1.9, 0.9 Hz, 1H), 3.53 (ddd, J=8.8, 4.1, 2.1 Hz, 1H), 2.59 (ddd, J=13.9, 8.8, 6.7 Hz, 1H), 2.48 (ddd, J=11.6, 9.8, 3.4 Hz, 1H), 2.33-2.16 (m, 2H), 2.13-2.10 (m, 1H), 1.89-1.71 (m, 5H), 1.53 (s, 3H), 1.51-1.42 (m, 2H), 1.38-1.27 (m, 4H), 0.89 (t, J=7.0 Hz, 3H)¹³C NMR (101 MHz, CDCl₃) δ 156.5, 154.8, 147.8, 144.1, 139.9, 124.9, 120.0, 111.6, 108.8, 102.3, 45.1, 40.2, 34.1, 32.0, 31.2, 30.4, 28.2, 23.8, 22.7, 21.5, 14.2.

### Example 9: Synthesis of I-2

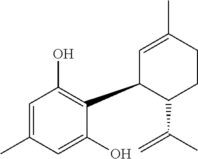

Synthesized following General Procedure A. (1S,4R)-1-Methyl-4-(1-methylethenyl)-2-cyclohexen-1-ol (0.050 g, 0.33 mmol), orcinol (0.12 g, 0.99 mmol), and alumina (0.66 g) in DCE (1.6 mL). The reaction was complete after 3 h of heating. Product was isolated as a colourless oil (0.51 g, 53%) by column chromatography gradient elution 0-25% ethyl acetate:hexanes. ¹H NMR (400 MHz, CDCl₃) δ 6.23-6.20 (m, 2H), 6.11 (s, 1H), 5.54 (s, 1H), 5.20 (brs, 1H), 4.65 (brt, J=1.7 Hz, 1H), 4.47 (s, 1H), 3.37-3.53 (m, 1H), 2.45 (ddd, J=11.4, 9.8, 3.4 Hz, 1H), 2.27-2.19 (m, 1H), 2.14 (s, 3H), 2.12-2.10 (m, 1H), 1.86-1.71 (m, 5H), 1.57 (s, 3H)¹³C NMR (101 MHz, CDCl₃) δ 156.4, 154.6, 147.7, 139.9, 139.1, 124.6, 120.5, 111.6, 109.7, 102.2, 45.2, 40.3, 30.3, 28.1, 23.7, 21.2, 21.0.

### Example 10: Synthesis of I-3

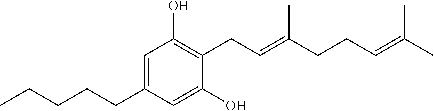 [SMILES: CCCCCc1cc(O)c(C/C=C(\C)CCC=C(C)C)c(O)c1]

Synthesized following General Procedure A. linalool (1.0 g, 6.5 mmol), olivetol (3.5 g, 19.4 mmol), and alumina (6.5 g) in DCE (33 mL). The reaction was complete after 3 h of heating. The product was isolated as a white solid (0.74 g, 30%) by column chromatography, gradient elution 0-25% ethyl acetate:hexanes. ¹H NMR (400 MHz, CDCl₃) δ 6.25 (s, 2H), 5.29-5.25 (m, 1H), 5.07-5.03 (m, 1H), 4.94 (s, 2H), 3.39 (d, J=7.1 Hz, 2H), 2.46 (app. t, J=7.6 Hz, 2H), 2.11-2.04 (m, 4H), 1.81 (s, 3H), 1.68 (s, 3H), 1.60-1.53 (m, 5H), 1.38-1.25 (m, 4H), 0.89 (t, J=6.9 Hz, 3H). ¹³C NMR (101 MHz, CDCl₃) δ 154.9, 142.9, 139.1, 132.2, 123.9, 121.8, 110.7, 108.5, 39.8, 35.7, 31.6, 30.9, 26.5, 25.8, 22.7, 22.4, 17.8, 16.3, 14.2.

### Example 11: Synthesis of I-4

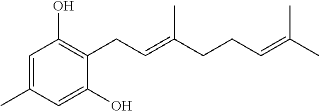 [SMILES: CC(C)=CCC/C(C)=C/Cc1c(O)cc(C)cc1O]

Synthesized following General Procedure A. linalool (0.1 g, 0.65 mmol), 3,5-dimethylphenol (0.24 g, 1.95 mmol), and alumina (1.3 g) in DCE (3 mL). The reaction was complete after 3 h of heating. The product was isolated as a pale-yellow oil (0.06 g, 34%) by column chromatography, gradient elution 0-25% ethyl acetate:hexanes. ¹H NMR (400 MHz, CDCl₃) δ 6.62 (s, 1H), 6.54 (s, 1H), 5.22-5.17 (m, 1H), 5.11-5.06 (m, 2H), 3.37 (d, J=6.9 Hz, 2H), 2.29 (s, 3H), 2.27 (s, 3H), 2.17-2.04 (m, 4H), 1.83 (s, 3H), 1.71 (s, 3H), 1.63 (s, 3H). ¹³C NMR (101 MHz, CDCl₃) δ 154.4, 137.5, 137.4, 136.6, 131.8, 124.1, 123.6, 122.7, 122.1, 114.4, 39.8, 26.6, 25.7, 25.4, 21.0, 19.9, 17.8, 16.2.

### Example 12: Synthesis of I-5

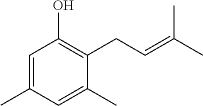

Synthesized following General Procedure A. 2-methylbut-3-en-2-ol (0.050 g, 0.58 mmol), 3,5-dimethylphenol (0.21 g, 1.74 mmol), and alumina (1.2 g) in DCE (3 mL). The reaction was complete after 4 h of heating. The product was isolated as a pale yellow oil (0.047 g, 42%) by column chromatography, gradient elution 0-25% ethyl acetate:hexanes. ¹H NMR (400 MHz, CDCl₃) δ 6.62 (s, 1H), 6.53 (s, 1H), 5.21-5.17 (m, 1H), 5.06 (s, 1H), 3.37 (d, J=6.9 Hz, 2H), 3.30 (s, 3H), 3.27 (s, 3H), 1.84 (s, 3H), 1.76 (s, 3H)¹³C NMR (101 MHz, CDCl₃) δ 154.2, 137.3, 136.6, 133.6, 123.6, 122.7, 122.1, 114.3, 25.8, 25.5, 21.0, 19.9, 17.9.

### Example 13: Synthesis of I-6

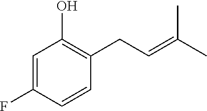

Synthesized following General Procedure A. 2-methylbut-3-en-2-ol (0.050 g, 0.58 mmol), 3-fluorophenol (0.19 g, 1.74 mmol), and alumina (1.2 g) in DCE (3 mL). The reaction was complete after 3 h of heating. The product was isolated as a pale yellow oil (0.018 g, 17%) by column chromatography, gradient elution 0-25% ethyl acetate:hexanes.

¹H NMR (400 MHz, CDCl₃) δ 7.04-7.00 (m, 1H), 6.59-6.53 (m, 2H), 5.33-5.26 (m, 2H), 3.31 (d, J=7.2 Hz, 2H), 1.78 (brs, 6H)¹³C NMR (101 MHz, CDCl₃) δ 163.5, 162.3 (d, ¹J_{CF}=243.4 Hz) 155.5, 155.4 (d, ³J_{CF}=11.4 Hz), 135.5, 130.7, 130.6 (d, ³J_{CF}=9.7 Hz), 122.5, 122.4 (d, ⁴J_{CF}=3.2 Hz), 121.6, 107.4, 107.3 (d, ²J_{CF}=21.0 Hz), 103.6, 103.5 (d, ²J_{CF}=24.4 Hz), 29.4, 25.9, 18.0 ¹⁹F {¹H} NMR (377 MHz, CDCl₃) 5-115.4

### Example 14: Synthesis of I-7

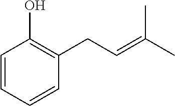

Synthesized following General Procedure A. 2-methylbut-3-en-2-ol (0.050 g, 0.58 mmol), phenol (0.16 g, 1.74 mmol), and alumina (1.2 g) in DCE (3 mL). The reaction was complete after 5 h of heating. The product was isolated as a colourless oil (0.033 g, 35%) by column chromatography, gradient elution 0-25% ethyl acetate:hexanes. ¹H NMR (400 MHz, CDCl₃) δ 7.15-7.10 (m, 2H), 6.90-6.86 (m, 1H), 6.83-6.80 (m, 1H), 5.37-5.32 (m, 1H), 5.20 (s, 1H), 3.38 (d, J=7.3 Hz, 2H), 1.80 (brs, 6H). ¹³C NMR (101 MHz, CDCl₃) δ 154.3, 134.8, 130.0, 127.6, 126.9, 121.9, 120.8, 115.8, 29.8, 25.9, 17.9.

### Example 15: Synthesis of I-8

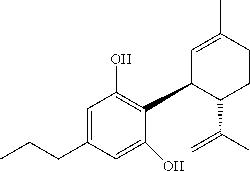

Synthesized following General Procedure A. (1S,4R)-1-Methyl-4-(1-methylethenyl)-2-cyclohexen-1-ol (0.100 g, 0.657 mmol), divarinol (0.30 g, 1.97 mmol), and alumina (1.31 g) in DCE (3 mL). The reaction was complete after 2 h of heating. The product was isolated as a yellow oil (0.097 g, 52%) by column chromatography, gradient elution 0-5% ethyl acetate:hexanes. R_{f} (20% ethyl acetate:hexanes) 0.23. ¹H NMR (400 MHz, Chloroform-d) δ 6.25-6.17 (m, 2H), 5.60 (s, 1H), 5.52 (dt, J=2.7, 1.5 Hz, 1H), 4.64 (t, J=1.7 Hz, 1H), 4.46 (dt, J=2.0, 0.9 Hz, 1H), 3.53 (ddd, J=9.6, 4.0, 2.2 Hz, 1H), 2.58 (ddd, J=13.8, 9.3, 6.0 Hz, 1H), 2.48 (ddd, J=11.6, 9.9, 3.5 Hz, 1H), 2.33-2.17 (m, 2H), 1.87-1.74 (m, 5H overlapped —CH₃ at 1.78, s), 1.53 (s, 3H), 1.56-1.45 (m, 2H), 0.92 (t, J=7.4 Hz, 3H)¹³C NMR (101 MHz, CDCl₃) δ 171.9, 156.3, 154.8, 147.7, 143.7, 139.8, 124.8, 119.8, 111.4, 108.8, 102.3, 102.2, 60.7, 45.0, 40.1, 36.1, 30.3, 28.2, 24.5, 23.6, 21.3, 21.1, 14.2, 14.2.

### Example 16: Synthesis of I-9

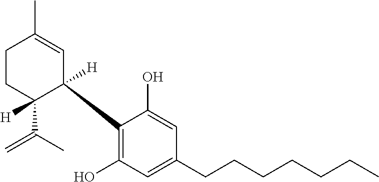 [SMILES: C=C(C)[C@@H]1CCC(C)=C[C@H]1c1c(O)cc(CCCCCCC)cc1O]

Synthesized following General Procedure A. (1S,4R)-1-Methyl-4-(1-methylethenyl)-2-cyclohexen-1-ol (0.061 g, 0.4 mmol), spherophorol (0.25 g, 1.2 mmol), and alumina (0.8 g) in cyclohexane (2 mL). The reaction was complete after 2 h of heating. The product was isolated as a yellow oil (0.035 g, 31%) by column chromatography, gradient elution 0-25% ethyl acetate:hexanes. R_{f} (20% ethyl acetate:hexanes) 0.32. ¹H NMR (400 MHz, Chloroform-d) δ 6.21 (q, J=2.7 Hz, 2H), 6.07 (s, 1H), 5.53 (br s, 1H), 4.88 (br s, 1H), 4.66-4.64 (m, 1H), 4.46 (br s, 1H), 3.53 (ddq, J=8.9, 4.5, 2.4 Hz, 1H), 2.62-2.55 (m, 1H), 2.48 (ddd, J=11.6, 9.8, 3.4 Hz, 1H), 2.30-2.18 (m, 2H), 2.13-2.06 (m, 1H), 1.87-1.72 (m, 5H), 1.53 (s, 3H), 1.50-1.42 (m, 2H), 1.32-1.25 (m, 8H), 0.88 (t, J=7.0 Hz, 3H) ¹³C NMR (101 MHz, CDCl₃) δ 156.5, 154.7, 147.7, 144.1, 139.9, 124.8, 120.0, 111.5, 108.7, 102.2, 45.1, 40.1, 34.1, 31.9, 31.5, 30.3, 29.8, 29.3, 28.2, 23.7, 22.8, 21.4, 14.2.

### Example 17: Synthesis of I-10

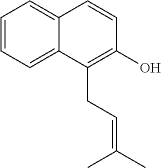 [SMILES: CC(C)=CCc1c(O)ccc2ccccc12]

Synthesized following General Procedure A. 2-Methyl-3-buten-2-ol (0.10 g, 1.2 mmol), 2-napthol (0.52 g, 3.6 mmol), and alumina (2.45 g) in DCE (6 mL). The reaction was complete after 2 h of heating. The product was isolated as a brown solid (0.18 g, 75%) by column chromatography, gradient elution 0-5% ethyl acetate:hexanes. R_{f} (20% ethyl acetate:hexanes) 0.69. ¹H NMR (400 MHz, Chloroform-d) δ 7.93 (d, J=8.6 Hz, 1H), 7.78 (d, J=8.1 Hz, 1H), 7.65 (d, J=8.8 Hz, 1H), 7.49 (ddd, J=8.4, 6.8, 1.4 Hz, 1H), 7.34 (ddd, J=8.0, 6.8, 1.1 Hz, 1H), 7.09 (d, J=8.7 Hz, 1H), 5.30-5.25 (m, 2H, overlapping with phenol —OH), 3.78 (d, J=6.8 Hz, 2H), 1.91 (d, J=1.4 Hz, 3H), 1.75 (d, J=1.5 Hz, 3H). ¹³C NMR (101 MHz, CDCl₃) δ 151.4, 134.3, 133.2, 129.6, 128.8, 128.1, 126.5, 123.2, 123.1, 122.3, 118.7, 118.2, 25.9, 24.6, 18.2.

### Example 18: Synthesis of I-11

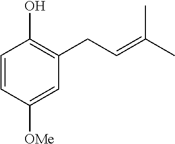

Synthesized following General Procedure A. 2-Methyl-3-buten-2-ol (0.1 g, 1.16 mmol), 4-methoxyphenol (0.43 g, 3.48 mmol), and alumina (2.32 g) in DCE (6 mL). The reaction was complete after 4.5 h of heating. The product was isolated as a yellow solid (0.070 g, 31%) by column chromatography, gradient elution 0-25% ethyl acetate:hexanes. R_{f} (20% ethyl acetate:hexanes) 0.43. ¹H NMR (400 MHz, CDCl₃) δ 6.78-6.64 (m, 3H), 5.32 (ddt, J=7.1, 5.2, 2.1 Hz, 1H), 4.91 (br. s, 1H) 3.76 (s, 3H), 3.33 (d, J=7.3 Hz, 2H), 1.78 (s, 6H). ¹³C NMR (101 MHz, CDCl₃) δ 153.7, 148.3, 134.8, 128.3, 121.7, 116.3, 115.8, 112.1, 55.8, 43.6, 30.0, 25.9, 18.0.

### Example 19: Synthesis of I-12 and I-13

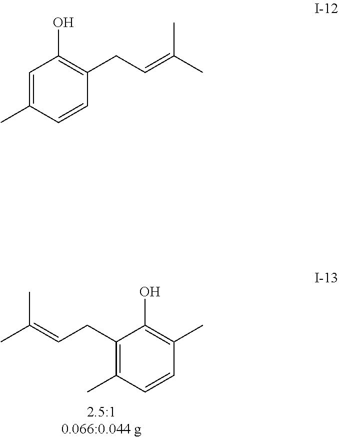

Synthesized following General Procedure A. 2-Methyl-3-buten-2-ol (0.12 mL, 1.2 mmol), m-cresol (0.38 g, 3.60 mmol), and alumina (2.32 g) in DCE (6 mL). The reaction was complete after 3.5 h of heating. The product was isolated as a 2.5:1 mixture of regioisomers 1-12 and I-13 (0.11 g, 52% overall yield), pale yellow oil. Purified by column chromatography, gradient elution 0-10% ethyl acetate:hexanes. R_{f} (20% ethyl acetate:hexanes) 0.60. ¹H NMR (400 MHz, CDCl₃) mixture of isomers, Major δ 7.00 (d, J=7.7 Hz, 1H), 6.70 (d, J=7.5 Hz, 1H), 6.65 (s, 1H), 5.33 (m, 1H), 5.07 (br s, 1H), 3.33 (d, J=7.3 Hz, 2H), 2.29 (s, 3H), 1.79 (s, 3H), 1.78 (s, 3H) Minor δ 7.03-7.01 (m, overlapped, 1H), 6.77 (d, J=5.18, 1H), 6.72-6.62 (m, overlapped, 1H), (t, J=6.9 Hz, 1H), 1.83 (s, 3H), 1.75 (s, 3H), 2.32 (s, 3H). ¹³C NMR (101 MHz, CDCl₃) mixture of isomers δ 154.2, 137.6, 134.6, 129.9, 126.8, 123.8, 122.8, 122.2, 121.8, 121.6, 116.6, 113.6, 29.6, 25.9, 21.1, 20.0, 18.0.

### Example 20: Synthesis of I-14

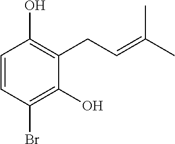

Synthesized following General Procedure A. 2-Methyl-3-buten-2-ol (0.13 mL, 1.2 mmol), 4-bromoresorcinol (0.68 g, 3.60 mmol), and alumina (2.32 g) in ethyl acetate (6 mL). The reaction was complete after 3.5 h of heating. The product was isolated as a brown solid (0.040 g, 13%) by column chromatography, gradient elution 0-20% ethyl acetate:hexanes. R_{f} (20% ethyl acetate:hexanes) 0.42. ¹H NMR (400 MHz, Chloroform-d) δ 7.17 (d, J=8.7 Hz, 1H), 6.35 (d, J=8.7 Hz, 1H), 5.60 (s, 1H), 5.31 (s, 1H), 5.25 (tdq, J=7.1, 2.8, 1.4 Hz, 1H), 3.46 (d, J=7.1 Hz, 2H), 1.83-1.81 (m, 3H), 1.75 (d, J=1.4 Hz, 3H). ¹³C NMR (101 MHz, CDCl₃) δ 155.2, 150.7, 135.4, 129.4, 121.2, 115.1, 109.7, 101.6, 25.9, 23.6, 18.0.

### Example 21: Synthesis of I-15

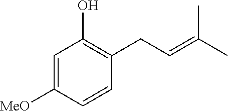

Synthesized following General Procedure A. 2-methylbut-3-en-2-ol (0.050 g, 0.37 mmol), 3-methoxyphenol (0.20 g, 1.12 mmol), and alumina (0.74 g) in DCE (2 mL). The reaction was complete after 3 h of heating. The product was isolated in a 29% yield by column chromatography, gradient elution 0-20% ethyl acetate:hexanes. ¹H NMR (400 MHz, Chloroform-d) δ 6.99 (dd, J=8.0, 0.7 Hz, 1H), 6.47-6.40 (m, 2H), 5.24 (br s, 1H), 5.31 (dddd, J=7.2, 5.8, 2.9, 1.4 Hz, 1H), 3.76 (s, 3H), 1.77 (dd, J=2.8, 1.4 Hz, 6H).

### Example 22: Synthesis of I-16

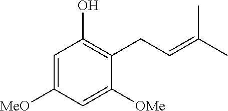 [SMILES: COc1cc(O)c(CC=C(C)C)c(OC)c1]

Synthesized following General Procedure A. 2-methylbut-3-en-2-ol (0.10 g, 1.2 mmol), 3,5-dimethoxyphenol (0.56 g, 3.6 mmol), and alumina (2.45 g) in DCE (6 mL). The reaction was complete after 2 h of heating. The product was isolated as a colourless oil (0.19, 69%) by column chromatography, gradient elution 0-5% ethyl acetate:hexanes. R_{f} (20% ethyl acetate:hexanes) 0.29. ¹H NMR (400 MHz, Chloroform-d) δ 6.14-6.04 (m, 2H), 5.22 (tdt, J=7.1, 2.9, 1.4 Hz, 1H), 3.78 (s, 3H), 3.75 (s, 3H), 3.33 (dt, J=7.2, 1.2 Hz, 2H), 1.80 (d, J=1.4 Hz, 3H), 1.73 (q, J=1.4 Hz, 3H). ¹³C NMR (101 MHz, CDCl₃) δ 159.5, 158.6, 156.2, 134.3, 122.6, 107.5, 93.8, 91.6, 55.8, 55.4, 25.9, 22.0, 17.9.

### Example 23: Synthesis of I-17

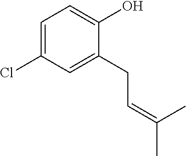

Synthesized following General Procedure A. 2-methylbut-3-en-2-ol (0.10 g, 1.2 mmol), p-chlorophenol (0.46 g, 3.6 mmol), and alumina (2.42 g) in DCE (6 mL). The reaction was complete after 18 h of heating. The product was isolated as a colourless oil (0.13 g, 58%) by column chromatography, gradient elution 0-5% ethyl acetate:hexanes. R_{f}(20% ethyl acetate:hexanes) 0.61. ¹H NMR (400 MHz, Chloroform-d) δ 7.11-7.02 (m, 2H), 6.72 (d, J=8.4 Hz, 1H), 5.31-5.72 (overlapped with phenol —OH, m, 2H), 3.32 (d, J=7.3 Hz, 2H), 1.79 (s, 3H), 1.77 (s, 3H). ¹³C NMR (101 MHz, CDCl₃) δ 152.9, 135.7, 129.7, 128.9, 127.2, 125.5, 121.0, 117.0, 29.5, 25.9, 18.0.

### Example 24: Synthesis of I-18

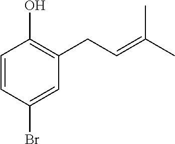

Synthesized following General Procedure A. 2-methylbut-3-en-2-ol (0.10 g, 1.2 mmol), p-bromophenol (0.63 g, 3.6 mmol), and alumina (2.43 g) in DCE (6 mL). The reaction was complete after 2 h of heating. The product was isolated as a mix with starting phenol (0.13 g, 45%) by column chromatography, gradient elution 0-5% ethyl acetate:hexanes. R_{f} (20% ethyl acetate:hexanes) 0.34. ¹H NMR (400 MHz, Chloroform-d) δ 7.23-7.17 (m, 2H), 6.68 (d, J=8.2 Hz, 1H), 5.28 (tdt, J=7.3, 2.9, 1.5 Hz, 1H), 5.12 (br s, 1H), 3.31 (d, J=7.3 Hz, 2H), 1.78 (d, J=1.2 Hz, 3H), 1.77 (s, 3H). ¹³C NMR (101 MHz, CDCl₃) δ 153.5, 135.8, 132.6, 130.3, 129.3, 120.9, 117.5, 117.3, 112.9, 29.6, 25.9, 18.0.

### Example 25: Synthesis of I-19

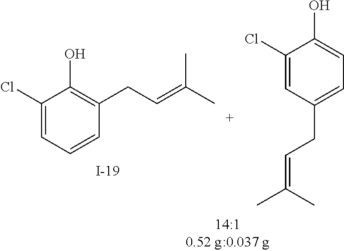

Synthesized following General Procedure A. 2-methylbut-3-en-2-ol (0.10 g, 1.2 mmol), o-chlorophenol (0.36 g, 3.5 mmol), and alumina (2.34 g) in DCE (6 mL). The reaction was complete after 3 h of heating. The desired product was isolated as a single spot by column chromatography, gradient elution 0-5% ethyl acetate:hexanes, that was a 14:1 mixture of the ortho and para substituted product (brown oil). 0.52 g, 22% yield ortho product. R_{f} (20% ethyl acetate:hexanes) 0.81. ¹H NMR (400 MHz, Chloroform-d) δ 7.18 (dd, J=8.1, 1.6 Hz, 1H), 7.05 (dd, J=7.7, 1.6 Hz, 1H), 6.80 (t, J=7.8 Hz, 1H), 5.65 (s, 1H), 5.33 (tdt, J=7.3, 2.9, 1.4 Hz, 1H), 3.39 (d, J=7.4 Hz, 2H), 1.77 (s, 3H), 1.75 (s, 3H). ¹³C NMR (101 MHz, CDCl₃) δ 149.4, 133.7, 128.5, 126.6, 121.7, 120.9, 114.0, 66.2, 29.1, 25.9, 17.9.

### Example 26: Synthesis of I-20

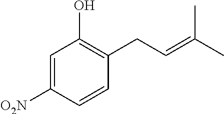

Synthesized following General Procedure A. 2-methylbut-3-en-2-ol (0.1 g, 1.16 mmol), 3-nitrophenol (0.3 g, 2.15 mmol), and alumina (2.32 g) in DCE (6 mL). The reaction was complete after 3 h of heating. The product was isolated as a brown oil (0.016 g, 7%), by column chromatography, gradient elution 0-10% ethyl acetate:hexanes. R_{f} (10% ethyl acetate:hexanes) 0.24. ¹H NMR (400 MHz, acetone-d⁶) δ 9.22 (s, 1H), 7.70-7.67 (m, 2H), 7.36-7.34 (m, 1H), 5.37-5.32 (m, 1H), 3.42 (d, J=7.4 Hz, 2H), 1.73 (s, 3H), 1.72 (s, 3H).

### Example 27: Synthesis of I-21

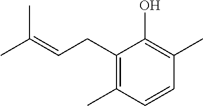

Synthesized following General Procedure A. 2-methylbut-3-en-2-ol (0.1 g, 1.2 mmol), 2,5-dimethylphenol (0.42 g, 3.5 mmol), and alumina (2.32 g) in DCE (6 mL). The reaction was complete after 2 h of heating. The product was isolated as a white solid (0.073 g, 33%) by column chromatography, gradient elution 0-25% ethyl acetate:hexanes. ¹H NMR (400 MHz, CDCl₃) δ 6.89 (d, J=7.6 Hz, 1H), 6.67 (d, J=7.6 Hz, 1H), 5.19-5.14 (m, 1H), 5.07 (s, 1H), 3.37 (d, J=6.9 Hz, 2H), 2.27 (s, 3H), 2.20 (s, 3H), 1.83 (s, 3H), 1.74 (s, 3H). ¹³C NMR (101 MHz, CDCl₃) δ 152.7, 134.8, 134.2, 128.2, 125.0, 122.1, 121.9, 121.7, 26.1, 25.8, 19.9, 18.0, 15.9.

### Example 28: Synthesis of I-22

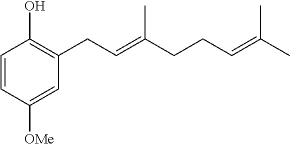 [SMILES: COc1ccc(O)c(C/C=C(\C)CCC=C(C)C)c1]

Synthesized following General Procedure A. Linalool (0.12 mL, 0.65 mmol), 4-methoxyphenol (0.24 g, 1.95 mmol), and alumina (1.30 g) in DCE (3.3 mL). The reaction was complete after 24 h of heating. The product was isolated as a pale yellow oil (0.056 g, 33%) by column chromatography, gradient elution 0-10% ethyl acetate:hexanes. R_{f} (20% ethyl acetate:hexanes) 0.58. ¹H NMR (400 MHz, CDCl₃) δ 6.75-6.64 (m, 3H), 5.31 (t, J=7.2 Hz, 1H), 5.08 (m, 1H), 4.80 (br s, 1H), 3.76 (s, 3H), 3.34 (d, J=7.2 Hz, 2H), 2.22-2.04 (m, 4H), 1.77 (s, 3H), 1.69 (s, 3H), 1.60 (s, 3H). ¹³C NMR (101 MHz, CDCl₃) δ 153.8, 148.4, 138.7, 132.1, 128.2, 124.0, 121.6, 116.5, 115.9, 115.8, 112.2, 112.2, 55.8, 39.8, 30.1, 26.6, 25.8, 17.8, 16.3.

### Example 29: Synthesis of I-23

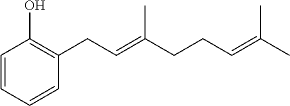 [SMILES: CC(C)=CCC/C(C)=C/Cc1ccccc1O]

Synthesized following General Procedure A. Linalool (0.10 g, 0.65 mmol), phenol (0.18 g, 1.95 mmol), and alumina (1.3 g) in DCE (3 mL). The reaction was complete after 3 h of heating. The product was isolated as a yellow oil (0.032 g, 10%) by column chromatography, gradient elution 0-25% ethyl acetate:hexanes. ¹H NMR (400 MHz, CDCl₃) b 7.13-7.05 (m, 4H), 5.39-5.33 (m, 1H), 5.31-5.27 (m, 1H), 3.38 (d, J=7.2 Hz, 2H), 2.23-2.07 (m, 4H), 1.78 (s, 3H), 1.70 (s, 3H), 1.62 (s, 3H). ¹³C NMR (101 MHz, CDCl₃) δ 154.5, 138.5, 132.0, 130.0, 127.5, 124.0, 121.8, 120.8, 115.8, 39.8, 29.7, 26.5, 25.8, 17.8, 16.2.

### Example 30: Synthesis of I-24

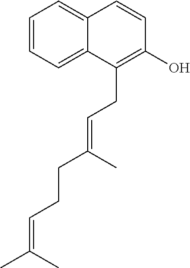 [SMILES: CC(C)=CCC/C(C)=C/Cc1c(O)ccc2ccccc12]

Synthesized following General Procedure A. Linalool (0.10 g, 1.2 mmol), 2-naphthol (0.52 g, 3.6 mmol), and alumina (2.44 g) in DCE (2 mL). The reaction was complete after 3 h of heating. The product was isolated as a brown solid (0.13 g, 59%) by column chromatography, gradient elution 0-5% ethyl acetate. R_{f} (20% ethyl acetate:hexanes) 0.45. ¹H NMR (400 MHz, Chloroform-d) δ 7.94 (d, J=8.6 Hz, 1H), 7.79 (d, J=8.1 Hz, 1H), 7.66 (d, J=8.8 Hz, 1H), 7.49 (t, J=7.7 Hz, 1H), 7.39-7.30 (m, 1H), 7.13-7.04 (m, 1H), 5.31-5.23 (m, 1H), 5.06 (tq, J=5.3, 1.6 Hz, 1H), 3.79 (d, J=6.7 Hz, 2H), 2.44-2.01 (m, 4H), 1.91 (s, 3H), 1.66 (s, 3H), 1.59 (s, 3H). ¹³C NMR (101 MHz, CDCl₃) δ 151.6, 138.1, 133.3, 132.0, 128.8, 128.1, 126.5, 126.5, 124.0, 123.2, 123.1, 122.2, 118.3, 39.8, 26.6, 25.8, 24.5, 17.8, 16.6.

### Example 31: Synthesis of I-25

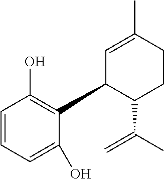

Synthesized following General Procedure A. (1S,4R)-1-Methyl-4-(1-methylethenyl)-2-cyclohexen-1-ol (0.10 g, 0.66 mmol), resorcinol (0.22 g, 1.97 mmol), and alumina (1.32 g) in DCE (3.3 mL). The reaction was complete after 4 h of heating. The product was isolated as a yellow solid (0.087 g, 55%) by column chromatography, gradient elution 0-15% ethyl acetate:hexanes. R_{f} (20% ethyl acetate:hexanes) 0.52. ¹H NMR (400 MHz, Chloroform-d) δ 7.15 (dd, J=8.4, 1.2 Hz, 1H), 6.38 (dd, J=8.4, 2.6 Hz, 1H), 6.29 (d, J=2.6 Hz, 1H), 5.89 (p, J=1.7 Hz, 1H), 4.69 (m, 1H), 3.11 (d, J=11.3 Hz, 1H), 2.14-2.08 (m, 2H), 1.92-1.81 (m, 2H), 1.73 (dd, J=2.4, 1.3 Hz, 3H), 1.48-1.30 (m, 4H) 1.16 (s, 3H). ¹³C NMR (101 MHz, CDCl₃) δ 155.0, 154.5, 134.9, 126.4, 122.4, 122.3, 108.0, 107.1, 103.9, 103.8, 44.8, 33.7, 31.0, 28.1, 24.7, 23.7, 21.0

### Example 32: Synthesis of I-26

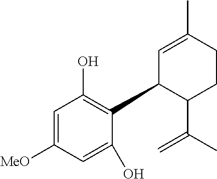 [SMILES: C=C(C)C1CCC(C)=C[C@@H]1c1c(O)cc(OC)cc1O]

Synthesized following General Procedure A. (1S,4R)-1-Methyl-4-(1-methylethenyl)-2-cyclohexen-1-ol (0.050 g, 0.37 mmol), 5-methoxybenzene-1,3-diol (0.20 g, 1.12 mmol), and alumina (0.74 g) in DCE (2 mL). The reaction was complete after 3 h of heating. The product was isolated as a colourless oil (0.036 g, 40%) by column chromatography, gradient elution 0-25% ethyl acetate:hexanes. ¹H NMR (400 MHz, CDCl₃) b 6.11 (brs, 1H), 5.96-5.94 (m, 2H), 5.55 (brs, 1H), 4.54 (brs, 1H), 4.49 (dd, J=2.4, 1.4 Hz, 1H), 4.33 (brs, 1H), 3.94-3.92 (m, 1H), 3.67 (s, 3H), 2.35 (td, J=10.4, 4.2 Hz, 1H), 2.24-2.20 (m, 1H), 2.10-2.09 (m, 1H), 1.79-1.75 (m, 5H), 1.65 (2, 3H).

### Example 33: Synthesis of I-27

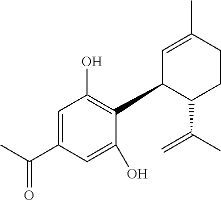

Synthesized following General Procedure A. (1S,4R)-1-Methyl-4-(1-methylethenyl)-2-cyclohexen-1-ol (0.10 g, 0.69 mmol), 3,5-dihydroxyacetophenone (0.30 g, 1.97 mmol), and alumina (1.35 g) in DCE (3 mL). The reaction was complete after 3 h of heating. The product was isolated a colourless oil (0.010 g, 5%) by column chromatography, gradient elution 0-5% ethyl acetate:hexanes. R_{f} (20% ethyl acetate:hexanes) 0.34. ¹H NMR (400 MHz, Chloroform-d) δ 6.59 (s, 1H), 6.40 (d, J=2.2 Hz, 1H), 6.20 (d, J=2.2 Hz, 1H), 5.64-5.55 (br m, 1H), 5.36 (s, 1H), 4.74 (s, 1H), 3.38 (dd, J=12.2, 1.7 Hz, 1H), 2.41 (dq, J=7.9, 2.1 Hz, 2H), 2.22 (d, J=1.6 Hz, 3H), 2.15 (d, J=1.8 Hz, 3H), 1.91 (t, J=1.2 Hz, 3H), 1.30-1.22 (m, 2H). ¹³C NMR (101 MHz, CDCl₃) δ 156.6, 153.2, 151.1, 142.8, 131.3, 130.8, 126.9, 100.7, 99.3, 60.5, 53.4, 47.2, 22.0, 12.6.

### Example 34: Synthesis of I-28

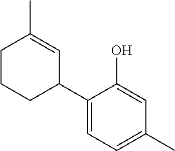

Synthesized following General Procedure A. 1-methylcyclohex-2-enol (0.050 g, 0.43 mmol), m-cresol (0.136 mL, 1.3 mmol), and alumina (0.87 g) in DCE (2 mL). The reaction was complete after 3 h of heating. The product was isolated as a yellow oil (0.022 g, 25%) by column chromatography, gradient elution 0-5% ethyl acetate:hexanes. R_{f} (20% ethyl acetate:hexanes) 0.71. ¹H NMR (400 MHz, Chloroform-d) δ 6.98 (d, J=7.6 Hz, 1H), 6.69 (dd, J=7.6, 1.8 Hz, 1H), 6.65 (d, J=1.7 Hz, 1H), 5.56 (dt, J=2.5, 1.3 Hz, 1H), 5.51 (s, 1H), 3.50-3.45 (m, 1H), 2.29 (s, 3H), 2.03-1.93 (m, 2H), 1.87-1.76 (m, 2H), 1.78 (s, 3H), 1.69-1.53 (m, 2H). ¹³C NMR (101 MHz, CDCl₃) δ 154.2, 138.9, 137.6, 129.6, 128.3, 123.9, 121.4, 117.0, 38.8, 30.1, 30.0, 24.2, 22.1, 21.1.

### Example 35: Synthesis of I-29

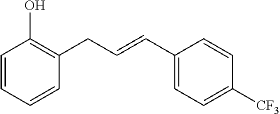 [SMILES: Oc1ccccc1C/C=C/c1ccc(C(F)(F)F)cc1]

Synthesized following General Procedure A. 1-(4-(trifluoromethyl)phenyl)prop-2-en-1-ol (0.04 g, 0.20 mmol), phenol (0.060 g, 0.59 mmol), and alumina (0.39 g) in DCE (1 mL). The reaction was complete after 5 h of heating. The product was isolated as a yellow solid (0.003 g, 6%) by column chromatography, gradient elution 0-15% ethyl acetate:hexanes. R_{f} (20% ethyl acetate:hexanes) 0.67. ¹H NMR (700 MHz, Chloroform-d) δ 7.58 (d, J=8.1 Hz, 1H), 7.50 (d, J=8.0 Hz, 2H), 7.33-7.29 (m, 2H), 7.01-6.94 (m, 3H), 6.78 (dd, J=16.1, 1.7 Hz, 1H), 6.52 (dt, J=16.0, 5.5 Hz, 1H), 4.73 (d, J=5.4 Hz, 1H). ¹⁹F NMR (659 MHz, CDCl₃) δ −62.54 (s, 3F).

### Example 36: Synthesis of I-30

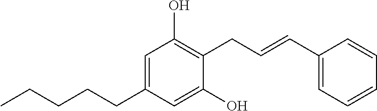 [SMILES: CCCCCc1cc(O)c(C/C=C/c2ccccc2)c(O)c1]

Synthesized following General Procedure A. 1-phenylprop-2-en-1-ol (0.050 g, 0.37 mmol), olivetol (0.20 g, 1.12 mmol), and alumina (0.74 g) in DCE (2 mL). The reaction was complete after 3 h of heating. The product was isolated as a white should (0.049 g, 44%) by column chromatography, gradient elution 0-20% ethyl acetate:hexanes. R_{f} (20% ethyl acetate:hexanes) 0.30. ¹H NMR (400 MHz, CDCl₃) δ 7.39-7.31 (m, 2H), 7.29-7.26 (m, 2H), 7.21-7.17 (m, 1H), 6.52 (dt, J=15.9, 1.7 Hz, 1H), 6.36 (dt, J=15.9, 6.3 Hz, 1H), 6.28 (s, 2H), 4.82 (s, 2H), 3.59 (dd, J=6.3, 1.6 Hz, 2H), 2.48 (t, J=7.5 Hz, 1H), 1.70-1.51 (m, 3H), 1.38-1.25 (m, 5H), 0.89 (t, J=6.9 Hz, 3H). ¹³C NMR (101 MHz, CDCl₃) δ 154.9, 143.4, 137.2, 131.1, 128.6, 127.8, 127.4, 126.4, 109.5, 108.6, 35.7, 31.6, 30.9, 26.7, 22.7, 14.2.

### Example 37: Synthesis of I-31

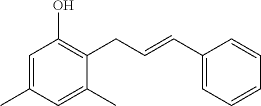 [SMILES: Cc1cc(C)c(C/C=C/c2ccccc2)c(O)c1]

Synthesized following General Procedure A. 1-phenylprop-2-en-1-ol (0.050 g, 0.37 mmol), 3,5-dimethylphenol (0.13 g, 1.11 mmol), and alumina (0.74 g) in DCE (2 mL). The reaction was complete after 2 h of heating. The product was isolated by column chromatography, gradient elution 0-10% ethyl acetate (0.043 g, 49%). R_{f} (10% ethyl acetate:hexanes) 0.28. ¹H NMR (400 MHz, CDCl₃) δ 7.33-7.24 (m, 4H), 7.20-7.15 (m, 1H), 6.63 (brs, 1H), 6.52 (brs, 1H), 6.40-6.28 (m, 2H), 4.69 (brs, 1H), 3.54 (d, J=5.2 Hz, 2H), 2.29 (s, 3H), 2.26 (s, 3H).

### Example 38: Synthesis of I-32

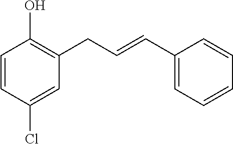 [SMILES: Oc1ccc(Cl)cc1C/C=C/c1ccccc1]

Synthesized following General Procedure A. 1-phenylprop-2-en-1-ol (0.050 g, 0.37 mmol), 4-chlorophenol (0.14 g, 1.12 mmol), and alumina (0.74 g) in DCE (2 mL). The reaction was complete after 3 h of heating. This product was collected as an inseparable mixture with 4-chlorophenol. The product was isolated as a yellow oil (˜0.054 g, 60%) mixed with starting phenol after column chromatography, gradient elution 0-20% ethyl acetate:hexanes. R_{f} (20% ethyl acetate:hexanes) 0.52. ¹H NMR (400 MHz, CDCl₃) δ 7.37-7.15 (m, overlapping with 4-chlorophenol), 6.76 (dd, J=8.7, 6.5 Hz, 2H), 6.51 (dt, J=15.9, 1.6 Hz, 1H), 6.34 (dt, J=15.9, 6.6 Hz, 1H), 4.99 (br s, 1H), 3.53 (dd, J=6.7, 1.6 Hz, 2H). mix with 4-chlorophenol ¹³C NMR (101 MHz, CDCl₃) δ 154.2, 152.7, 137.0, 132.2, 130.2, 129.7, 128.7, 127.7, 127.7, 127.7, 127.0, 126.4, 125.8, 125.8, 117.1*, 116.8*, 34.0* *—signal is certain to compound LI-01-026

### Example 39: Synthesis of I-33

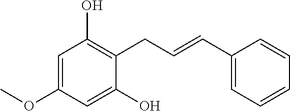 [SMILES: COc1cc(O)c(C/C=C/c2ccccc2)c(O)c1]

Synthesized following General Procedure A. 1-phenylprop-2-en-1-ol (0.050 g, 0.37 mmol), 5-methoxybenzene-1,3-diol (0.16 g, 1.12 mmol), and alumina (0.74 g) in DCE (2 mL). The reaction was complete after 3 h of heating. The product was isolated as a white solid (0.035 g, 71%) by column chromatography, gradient elution 0-40% ethyl acetate:hexanes. R_{f} (20% ethyl acetate:hexanes) 0.48. ¹H NMR (400 MHz, CDCl₃) δ 7.36-7.31 (m, 2H), 7.31-7.27 (m, 2H), 7.23-7.17 (m, 1H), 6.50 (dt, J=15.9, 1.7 Hz, 1H), 6.34 (dt, J=15.9, 6.2 Hz, 1H), 6.05 (s, 2H), 4.95 (br s, 2H), 3.74 (s, 3H), 3.55 (dd, J=6.2, 1.7 Hz, 2H). ¹³C NMR (101 MHz, CDCl₃) δ 159.7, 155.7, 137.1, 131.1, 128.7, 128.7, 127.9, 127.5, 126.4, 126.3, 104.7, 94.9, 55.5, 26.4

### Example 40: Synthesis of I-34 and I-35

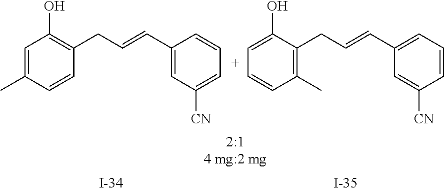

Synthesized following General Procedure A. 3-(1-hydroxyallyl)benzonitrile (0.024 g, 0.15 mmol), m-cresol (0.048 g, 0.44 mmol), and alumina (0.29 g) in DCE (0.75 mL). The reaction was complete after 19 h of heating. The product was isolated as a colourless oil and a 2:1 mixture of ortho regioisomers, 1-34 and I-35 (0.006 g, 17% combined yield) by column chromatography, gradient elution 0-40% ethyl acetate:hexanes. R_{f} (20% ethyl acetate:hexanes) 0.31. ¹H NMR (700 MHz, Chloroform-d) δ 7.60 (s, 1H), 7.54 (d, J=7.9 Hz, 1H), 7.46 (dd, J=12.4, 7.7 Hz, 1H), 7.38-7.34 (m, 1H), 7.03 (d, J=7.7 Hz, 1H), 6.73 (d, J=7.6 Hz, 1H), 6.63 (s, 1H), 6.49-6.43 (m, 1H), 6.41 (s, 1H), 3.54 (d, J=6.3 Hz, 2H), 2.30 (s, 3H). ¹³C NMR (176 MHz, CDCl₃) δ 153.6, 138.8, 138.3, 131.7, 130.5, 130.5, 130.4, 129.8, 129.4, 128.8, 122.0, 116.5, 113.3, 33.6, 21.2.

### Example 41: Synthesis of I-36 and I-37

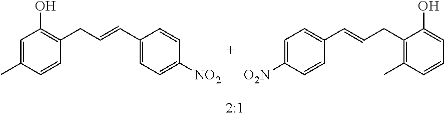 [SMILES: Cc1ccc(C/C=C/c2ccc([N+](=O)[O-])cc2)c(O)c1 ; Cc1cccc(O)c1C/C=C/c1ccc([N+](=O)[O-])cc1]

Synthesized following General Procedure A. 1-(4-nitrophenyl)prop-2-en-1-ol (0.013 g, 0.07 mmol), phenol (0.024 g, 0.23 mmol), and alumina (0.150 g) in DCE (0.5 mL). The reaction did not go to completion after 48 h. The product was isolated as a yellow solid and a mixture of ortho regioisomers 1-36 and I-37 (0.003 g, 17%), by column chromatography, gradient elution 0-20% ethyl acetate:hexanes. R_{f} (20% ethyl acetate:hexanes) 0.34. ¹H NMR (400 MHz, CDCl₃) δ 8.16-8.11 (m, 2H), 7.55-7.36 (m, 2H), 7.04 (d, J=7.6 Hz, 1H), 6.76-6.72 (m, 1H), 6.63-6.52 (m, 2H), 6.48-6.35 (m, 1H), 4.70 (br s, 1H), 3.57 (d, J=6.2 Hz, 2H), 2.30 (s, 3H).

### Example 42: Synthesis of I-38

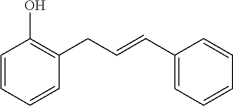 [SMILES: Oc1ccccc1C/C=C/c1ccccc1]

Synthesized following General Procedure A. 1-phenylprop-2-en-1-ol (0.050 g, 0.37 mmol), phenol (0.10 g, 1.12 mmol), and alumina (0.74 g) in DCE (2 mL). The reaction was complete after 18 h of heating. The product was isolated as a white solid (0.037 g, 47%) by column chromatography, gradient elution 0-30% ethyl acetate:hexanes. R_{f} (20% ethyl acetate:hexanes) 0.46. ¹H NMR (400 MHz, Chloroform-d) δ 7.41-7.34 (m, 2H), 7.33-7.27 (m, 2H), 7.25-7.11 (m, 3H), 6.92 (td, J=7.5, 1.2 Hz, 1H), 6.83 (dd, J=7.9, 1.2 Hz, 1H), 6.52 (dt, J=15.8, 1.5 Hz, 1H), 6.40 (dt, J=15.9, 6.4 Hz, 1H), 4.94 (s, 1H), 3.58 (dd, J=6.6, 1.4 Hz, 2H). ¹³C NMR (101 MHz, CDCl₃) δ 154.1, 137.2, 131.6, 130.6, 128.7, 128.1, 127.5, 126.3, 125.8, 121.2, 115.9, 34.2.

### Example 43: Synthesis of I-39

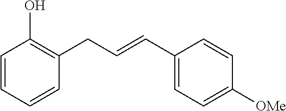 [SMILES: COc1ccc(/C=C/Cc2ccccc2O)cc1]

Synthesized following General Procedure A. 1-(4-methoxyphenyl)prop-2-en-1-ol (0.050 g, 0.30 mmol), phenol (0.086 g, 0.91 mmol), and alumina (0.60 g) in DCE (1.5 mL). The reaction was complete after 16 h of heating. The product was isolated as a white solid (0.013 g, 18%) by column chromatography, gradient elution 0-20% ethyl acetate:hexanes. R_{f} (20% ethyl acetate:hexanes) 0.26. ¹H NMR (400 MHz, CDCl₃) δ 7.38-7.22 (m, 2H), 7.20-7.07 (m, 2H), 6.91 (td, J=7.4, 1.2 Hz, 1H), 6.87-6.80 (m, 3H), 6.47 (dt, J=15.9, 1.6 Hz, 1H), 6.25 (dt, J=15.8, 6.6 Hz, 1H), 5.02 (s, 1H), 3.80 (s, 3H), 3.56 (dd, J=6.7, 1.6 Hz, 2H). ¹³C NMR (101 MHz, CDCl₃) δ 159.2, 154.3, 131.2, 130.6, 130.0, 128.0, 127.5, 125.9, 125.7, 121.1, 115.9, 114.10, 55.4, 34.3.

### Example 44: Synthesis of I-40

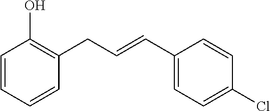 [SMILES: Oc1ccccc1C/C=C/c1ccc(Cl)cc1]

Synthesized following General Procedure A. 1-(4-chlorophenyl)prop-2-en-1-ol (0.050 g, 0.30 mmol), phenol (0.086 g, 0.89 mmol), and alumina (0.59 g) in DCE (1.5 mL). The reaction was complete after 16 h of heating. The product was isolated as a white solid (0.043 g, 60%) by column chromatography, gradient elution 0-20% ethyl acetate:hexanes. R_{f} (20% ethyl acetate:hexanes) 0.40. ¹H NMR (400 MHz, CDCl₃) δ 7.28-7.13 (m, 4H), 7.20-7.10 (m, 2H), 6.92 (tdd, J=7.4, 4.0, 2.3 Hz, 1H), 6.81 (dd, J=7.8, 1.9 Hz, 1H), 6.50-6.27 (m, 2H), 4.85 (br s, 1H), 3.56 (dd, J=5.8, 2.0 Hz, 2H). ¹³C NMR (101 MHz, CDCl₃) δ 154.0, 135.8, 133.0, 130.7, 130.6, 130.3, 128.9, 128.9, 128.8, 128.1, 127.5, 121.3, 121.2, 115.8, 34.1.

### Example 45: Synthesis of I-41

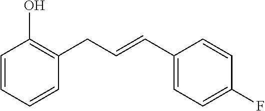 [SMILES: Oc1ccccc1C/C=C/c1ccc(F)cc1]

Synthesized following General Procedure A. 1-(4-fluorophenyl)prop-2-en-1-ol (0.050 g, 0.33 mmol), phenol (0.093 g, 0.98 mmol), and alumina (0.66 g) in DCE (1.6 mL). The reaction was complete after 18 h of heating. The product was isolated as an inseparable mixture with the starting phenol (pale yellow oil, 0.087 g mixture) by column chromatography, gradient elution 0-20% ethyl acetate:hexanes. R_{f} (20% ethyl acetate:hexanes) 0.41. ¹H NMR mixture with phenol (400 MHz, CDCl₃) δ 7.31-7.13 (overlapped with phenol), 6.99-6.90 (overlapped with phenol), 6.85-6.81 (overlapped with phenol), 6.44 (dt, J=15.8, 1.6 Hz, 1H), 6.30 (dt, J=15.8, 6.5 Hz, 1H), 5.24 (s, 1H), 3.56 (dd, J=6.6, 1.5 Hz, 2H). ¹³C NMR mix with phenol (101 MHz, CDCl₃) δ 154.7 (d, J=157.2 Hz), 130.44 (d, J=34.1 Hz), 129.77, 128.00, 127.84, 127.82, 127.75, 127.67, 121.18, 120.92, 115.82, 115.54, 115.41, 115.33, 33.92. ¹⁹F NMR (377 MHz, CDCl₃, {¹H}) δ −114.97 (s, 1F).

### Example 46: Synthesis of I-42

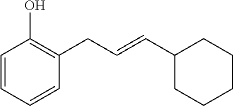 [SMILES: Oc1ccccc1C/C=C/C1CCCCC1]

Synthesized following General Procedure A. 1-cyclohexylprop-2-en-1-ol (0.050 g, 0.36 mmol), phenol (0.10 g, 1.07 mmol), and alumina (0.72 g) in DCE (2 mL). The reaction was not complete after 24 h of heating but was quenched at this point and product collected. The product was collected as a pale-yellow oil (0.014 g, 18%) by column chromatography, gradient elution 0-20% ethyl acetate:hexanes. R_{f} (20% ethyl acetate:hexanes) 0.53. ¹H NMR (400 MHz, CDCl₃) δ 7.14 (td, J=7.7, 1.7 Hz, 1H), 7.09 (dd, J=7.5, 1.7 Hz, 1H), 6.87 (td, J=7.4, 1.2 Hz, 1H), 6.82 (dd, J=8.0, 1.2 Hz, 1H), 5.68-5.61 (m, 1H), 5.57 (dt, J=15.6, 6.5 Hz, 1H), 5.20 (s, 1H), 3.36 (d, J=6.3 Hz, 2H), 1.98 (dp, J=11.3, 3.7, 3.3 Hz, 1H), 1.74-1.70 (m, 4H), 1.29-1.23 (m 3H), 1.18-1.05 (m, 3H). ¹³C NMR (176 MHz, CDCl₃) δ 154.6, 139.4, 130.2, 127.8, 125.7, 125.1, 120.7, 115.9, 40.5, 34.7, 32.9, 26.1, 25.9.

### Example 47: Synthesis of I-43

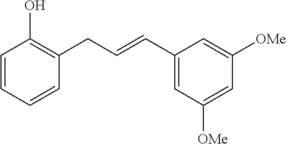 [SMILES: COc1cc(/C=C/Cc2ccccc2O)cc(OC)c1]

Synthesized following General Procedure A. 1-(3,5-dimethoxyphenyl)prop-2-en-1-ol (0.050 g, 0.26 mmol), phenol (0.073 g, 1.12 mmol), and alumina (0.51 g) in DCE (1.3 mL). The reaction was complete after 4 h of heating. The product was isolated as an off-white solid (0.036 g, 52%) by column chromatography, gradient elution 0-30% ethyl acetate:hexanes. R_{f} (20% ethyl acetate:hexanes) 0.28. ¹H NMR (400 MHz, CDCl₃) δ 7.20-7.12 (m, 2H), 6.92 (td, J=7.4, 1.2 Hz, 1H), 6.82 (dd, J=7.9, 1.2 Hz, 1H), 6.53 (d, J=2.3 Hz, 2H), 6.44-6.33 (m, 3H), 5.03 (s, 1H), 3.79 (s, 6H), 3.57 (d, J=5.6 Hz, 2H). ¹³C NMR (101 MHz, CDCl₃) δ 161.0, 154.1, 139.3, 131.5, 130.6, 128.7, 128.0, 125.8, 121.1, 115.9, 104.4, 99.9, 55.5, 55.4, 34.0.

### Example 48: Synthesis of I-44

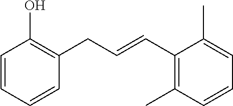 [SMILES: Cc1cccc(C)c1/C=C/Cc1ccccc1O]

Synthesized following General Procedure A. 1-(2,6-dimethylphenyl)prop-2-en-1-ol (0.050 g, 0.31 mmol), phenol (0.087 g, 0.93 mmol), and alumina (0.62 g) in DCE (1.6 mL). The reaction was complete after 4 h of heating. The product was isolated as a pale-yellow oil (0.046 g, 62%) by column chromatography, gradient elution 0-30% ethyl acetate:hexanes. R_{f} (20% ethyl acetate:hexanes) 0.50. ¹H NMR (400 MHz, CDCl₃) δ 7.23-7.12 (m, 2H), 7.08-6.99 (m, 3H), 6.92 (td, J=7.5, 1.2 Hz, 1H), 6.83 (dd, J=8.0, 1.2 Hz, 1H), 5.88 (dt, J=16.2, 6.6 Hz, 1H), 4.98 (br s, 1H), 3.62 (dd, J=6.5, 1.7 Hz, 2H). ¹³C NMR (101 MHz, CDCl₃) δ 154.1, 136.8, 135.9, 132.8, 130.4, 129.5, 127.9, 127.7, 126.5, 125.7, 121.0, 115.7, 34.6, 21.0.

### Example 49: Synthesis of I-45, I-51 and I-47

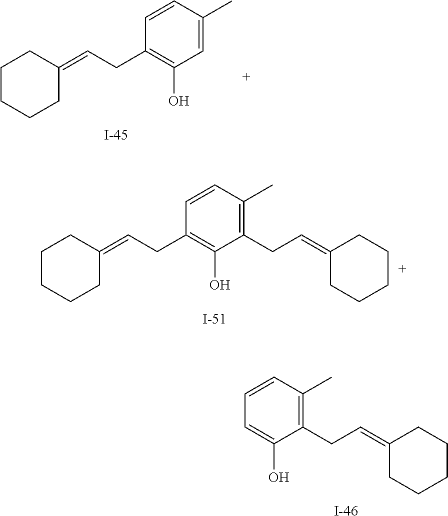

Synthesized following General Procedure A. 1-vinylcyclohexanol (0.10 g, 0.79 mmol), m-cresol (0.26 g, 2.38 mmol), and alumina (1.58 g) in DCE (4 mL). The reaction was complete after 4 h of heating. This product was isolated as a mixture of desired isomer, ortho-regioisomer and double addition. The mixture of isomers, 1-45, I-51 and I-47, was isolated as a colourless oil (0.074 g, 43% combined isomers) by column chromatography, gradient elution 0-20% ethyl acetate:hexanes. R_{f} (20% ethyl acetate:hexanes) 0.63. ¹H NMR (400 MHz, Chloroform-d) δ 7.00 (d, J=7.6 Hz, 1H), 6.69 (d, J=7.6 Hz, 1H), 6.65 (s, 1H), 5.27 (tt, J=7.3, 1.3 Hz, 1H), 5.18 (s, 1H), 3.35 (d, J=7.4 Hz, 2H), 2.29 (s, 3H), 2.18-2.10 (m, 2H), 1.62-1.55 (m, 8H). ¹³C NMR (101 MHz, CDCl₃) δ 154.4, 142.8, 137.7, 129.9, 121.6, 118.8, 116.6, 37.3, 28.8, 28.7, 27.8, 26.9, 21.1.

### Example 50: Synthesis of I-47, I-52 and I-48

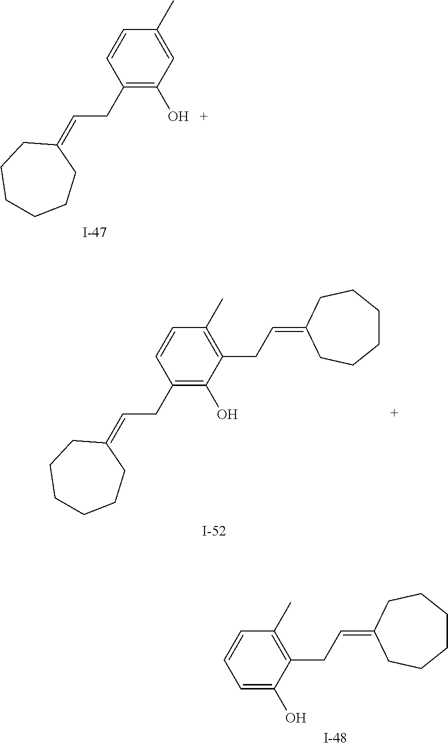

Synthesized following General Procedure A. 1-vinylcycloheptanol (0.10 g, 0.71 mmol), m-cresol (0.23 g, 2.14 mmol), and alumina (1.42 g) in DCE (3.6 mL). The reaction was complete after 2 h of heating. This product was isolated as a mixture of desired isomer, ortho-regioisomer and double addition. The mixture of isomers was isolated as a colourless oil (0.079 g, 48% combined isomers) by column chromatography, gradient elution 0-20% ethyl acetate:hexanes. R_{f} (20% ethyl acetate:hexanes) 0.48. ¹H NMR (400 MHz, Chloroform-d) δ 6.99 (d, J=7.6 Hz, 1H), 6.68 (d, J=7.8 Hz, 1H), 6.64 (s, 1H), 5.32 (tt, J=7.2, 1.4 Hz, 1H), 5.11 (s, 1H), 3.32 (d, J=7.1 Hz, 2H), 2.43-2.38 (m, 2H), 2.28 (s, 3H), 1.63-1.47 (m, 10H). ¹³C NMR (101 MHz, CDCl₃) δ 154.3, 144.8, 137.5, 129.7, 122.2, 121.4, 116.4, 113.6, 37.8, 30.3, 29.9, 29.3, 29.2, 29.1, 26.9, 21.0.

### Example 51: Synthesis of I-49 and I-50

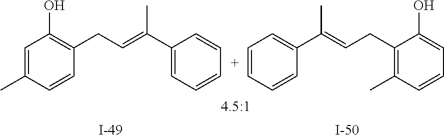

Synthesized following General Procedure A. 2-phenylbut-3-en-2-ol (0.05 g, 0.39 mmol), m-cresol (0.13 g, 1.17 mmol), and alumina (0.78 g) in acetonitrile (1.9 mL). The reaction was complete after 2 h of heating. The product was isolated as a mixture of ortho regioisomers as a pale yellow oil (0.007 g, 5%) by column chromatography, gradient elution 0-20% ethyl acetate:hexanes. Product is significantly overlapped with the starting phenol during chromatography and yield is lost to this mixture. R_{f} (20% ethyl acetate:hexanes) 0.47. ¹H NMR (700 MHz, Chloroform-d) δ 7.40 (d, J=7.6 Hz, 2H), 7.31 (t, J=7.7 Hz, 2H), 7.23 (t, J=7.5 Hz, 1H), 7.05 (d, J=7.6 Hz, 1H), 6.71 (d, J=7.6 Hz, 1H), 6.64 (s, 1H), 5.94 (t, J=7.2 Hz, 1H), 3.53 (d, J=7.3 Hz, 2H), 2.29 (s, 3H), 2.18 (s, 3H). ¹³C NMR (176 MHz, CDCl₃) δ 153.9, 143.5, 137.7, 136.9, 130.0, 128.4, 127.0, 125.9, 125.8, 121.8, 116.5, 29.7, 21.1, 16.1.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A process for preparing a compound of Formula (I): 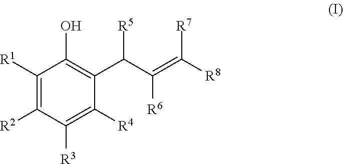
comprising reacting a compound of Formula (II): 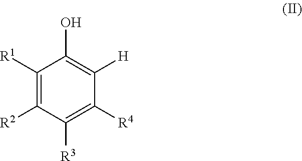
with a compound of Formula (III): 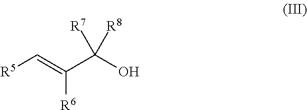
in presence of aluminum compound selected from alumina and aluminum alkoxides and in a non-protic solvent to form the compound of Formula (I),
wherein:
R¹, R², R³ and R⁴ are independently selected from H, OH, protected hydroxyl, halo, CN, NO₂, COOH, C₁₋₂₀alkyl, C₁₋₂₀haloalkyl, C₂₋₂₀alkenyl, C₂₋₂₀alkynyl, C₃₋₂₀cycloalkyl, C₃₋₂₀heterocycloalkyl, aryl, C₅₋₂₀heteroaryl, Z—C₁₋₂₀alkyl, Z—C₂₋₂₀alkenyl, Z—C₂₋₂₀alkynyl, Z—C₃₋₂₀cycloalkyl, Z—C₃₋₂₀heterocycloalkyl, Z-aryl, and Z—C₅₋₂₀heteroaryl, wherein the later 14 groups are unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl, or
R¹ and R², R² and R³ and/or R³ and R⁴ are linked together to form a polycyclic ring system having 8 or more atoms including the atoms in the phenyl ring to which said R¹, R², R³ and R⁴ groups are bonded, and in which one or more carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
Z is selected from O, C(O), CO₂, S, SO₂, SO, and NR¹⁰;
R⁵ is selected from H, C₁₋₆alkyl and C₁₋₆haloalkyl;
R⁶ is selected from H, C₁₋₆alkyl and C₁₋₆haloalkyl;
R⁷ is selected from C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁸ is selected from H, C₁₋₂₀alkyl, C₂₋₂₀alkenyl, C₂₋₂₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl; or
any R⁵ and R⁶, R⁵ and R⁷ or R⁷ and R⁸ are linked together to form an unsubstituted or substituted monocyclic or polycyclic ring system having 3 or more atoms together with the carbon atoms to which these groups are bonded and are therebetween, and in which one or more carbon atoms in the monocyclic or polycyclic ring system is optionally replaced with a heteromoiety selected from NR¹¹, O and S, and the monocyclic or polycyclic ring system is unsubstituted or substituted with one or more substituents independently selected from ═O, OH, NH₂, halo, NHC₁₋₂₀alkyl, N(C₁₋₂₀alkyl)(C₁₋₂₀alkyl), C₁₋₂₀alkyl, C₂₋₂₀alkenyl, C₂₋₂₀alkynyl, OC₁₋₂₀alkyl, OC₂₋₂₀alkenyl, OC₂₋₂₀alkynyl, SC₁₋₂₀alkyl, SC₂₋₂₀alkenyl, SC₂₋₂₀alkynyl, S(O)C₁₋₂₀alkyl, S(O)C₂₋₂₀alkenyl, S(O)C₂₋₂₀alkynyl, SO₂C₁₋₂₀alkyl, SO₂C₂₋₂₀alkenyl, SO₂C₂₋₂₀alkynyl, aryl, C₅₋₂₀heteroaryl, C₃₋₂₀cycloalkyl, and C₃₋₂₀heterocycloalkyl, the latter 21 groups being unsubstituted or further substituted with one or more substituents independently selected from OH, halo, C₁₋₂₀alkyl, C₁₋₂₀haloalkyl, C₂₋₂₀alkenyl, OC₁₋₂₀alkyl and OC₁₋₂₀haloalkyl;
R⁹, R¹⁰ and R¹¹ are independently selected from H, C₁₋₆alkyl and C₁₋₆haloalkyl.

2. The process of claim 1, wherein R¹, R², R³ and R⁴ are independently selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

3. (canceled)

4. The process of claim 23, wherein R¹ is selected from H, F, Cl, Br, CN, NO₂, C₁₋₁₀alkyl, C₁₋₁₀haloalkyl and Z—C₁₋₁₀alkyl; R² is selected from H, F, Cl, Br, CN, NO₂, C₁₋₁₀alkyl, C₁₋₁₀haloalkyl, C₂₋₁₀alkenyl and Z—C₁₋₁₀alkyl; and/or R⁴ is selected from H, OH, C₁₋₁₀alkyl, C₁₋₁₀haloalkyl and Z—C₁₋₁₀alkyl.

5. (canceled)

6. (canceled)

7. The process of claim 1, wherein Z is selected from SO₂, O, C(O) and NR¹⁰ or wherein Z is selected from O and C(O).

8. (canceled)

9. (canceled)

10. The process of claim 1, wherein R⁴ is OH and the compound of Formula (II) is 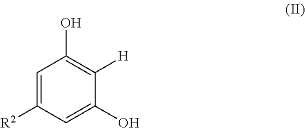
wherein:
R² is selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl;
Z is selected from O, C(O), CO₂, S, SO₂, SO, and NR¹⁰; and
R¹⁰ is selected from C₁₋₆alkyl and C₁₋₆fluoroalkyl.

11. (canceled)

12. (canceled)

13. The process of claim 1, wherein the compound of Formula (II) is selected from

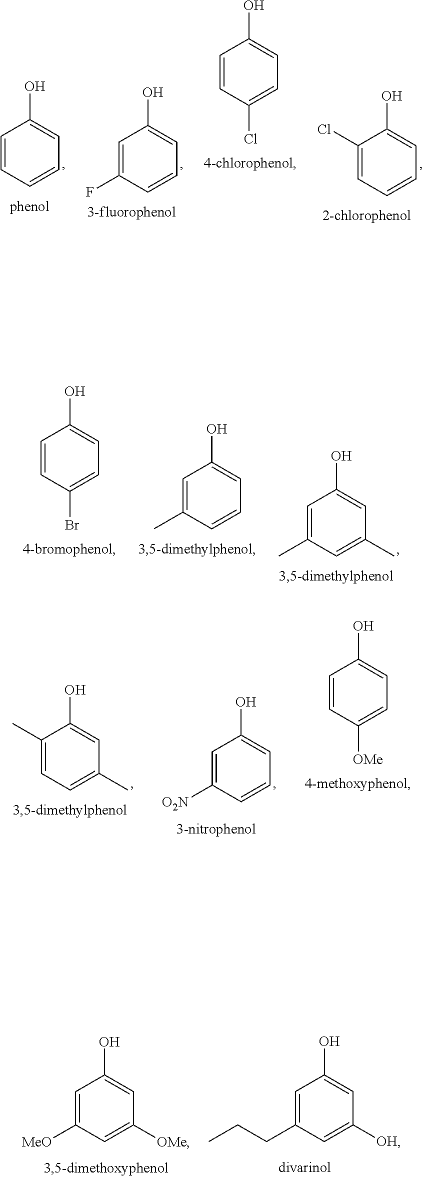

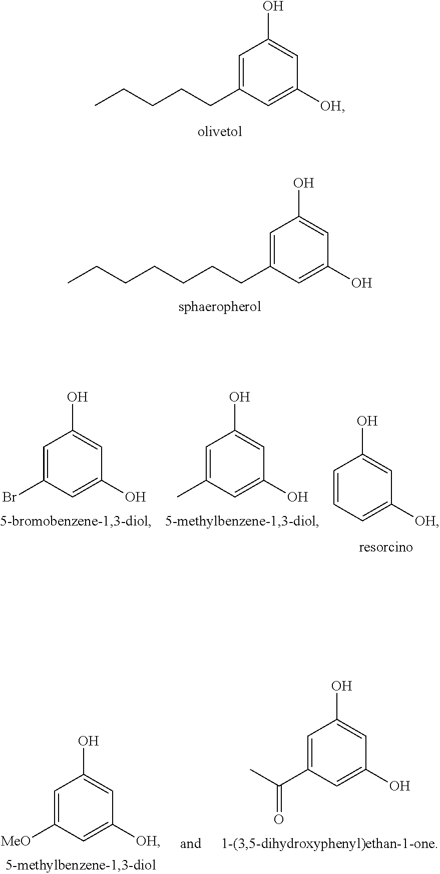

14. The process of claim 1, wherein R¹ and R², R² and R³ and/or R³ and R⁴ are linked together to form a polycyclic ring system having 10 or more atoms including the atoms in the phenyl ring to which said R¹, R², R³ and R⁴ groups are bonded, and in which one to three carbon atoms in said polycyclic ring system is optionally replaced with a heteromoiety selected from NR⁹, O and S, and the polycyclic ring system is unsubstituted or substituted with one or more substituents independently selected from ═O, OH, NH₂, halo, NHC₁₋₂₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₂₋₁₀alkenyl, OC₂₋₁₀alkynyl, SC₁₋₁₀alkyl, SC₂₋₁₀alkenyl, SC₂₋₁₀alkynyl, S(O)C₁₋₁₀alkyl, S(O)C₂₋₁₀alkenyl, S(O)C₂₋₁₀alkynyl, SO₂C₁₋₁₀alkyl, SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₁₀alkyl, C₁₋₁₀haloalkyl, C₂₋₁₀alkenyl, OC₁₋₁₀alkyl and OC₁₋₁₀haloalkyl.

15.-18. (canceled)

19. The process of claim 1, wherein the compound of Formula (III) has the following structure: 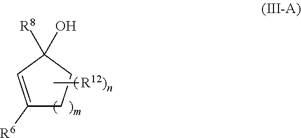
wherein:
R⁸ is selected from H, C₁₋₆alkyl and fluoroC₁₋₆alkyl;
R⁶ is selected from H, C₁₋₆alkyl and fluoroC₁₋₆alkyl;
each R¹² is independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl;
m is 1, 2, 3, 4, 5, 6, 7, or 8; and
n is 1, 2, 3 or 4.

20.-22. (canceled)

23. The process of claim 1, wherein the compound of Formula (III) has the following structure: 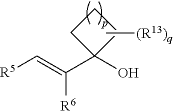
wherein
each R¹³ is independently selected from ═O, OH, NH₂, halo, CN, NO₂, COOH, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl;
p is 1, 2, 3, 4 or 5 and
q is 0 1, 2, 3 or 4.

24. (canceled)

25. The process of claim 1, wherein the compound of Formula (III) has the following structure: 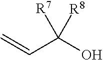
wherein:
R⁷ is selected from C₁₋₁₀alkyl, C₂₋₁₀alkenyl, C₂₋₁₀alkynyl, aryl, heteroaryl, cycloalkyl and heterocycloalkyl, each of which is unsubstituted or substituted with one or more substituents independently selected from ═O, OH, NH₂, halo, CN, NO₂, COOH, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl, C₂₋₆alkenyl, OC₁₋₆alkyl and OC₁₋₆fluoroalkyl; and
R⁸ is selected from H, C₁₋₆alkyl and fluoroC₁₋₆alkyl.

26.-29. (canceled)

30. The process of claim 1, wherein the compound of Formula (III) is selected from

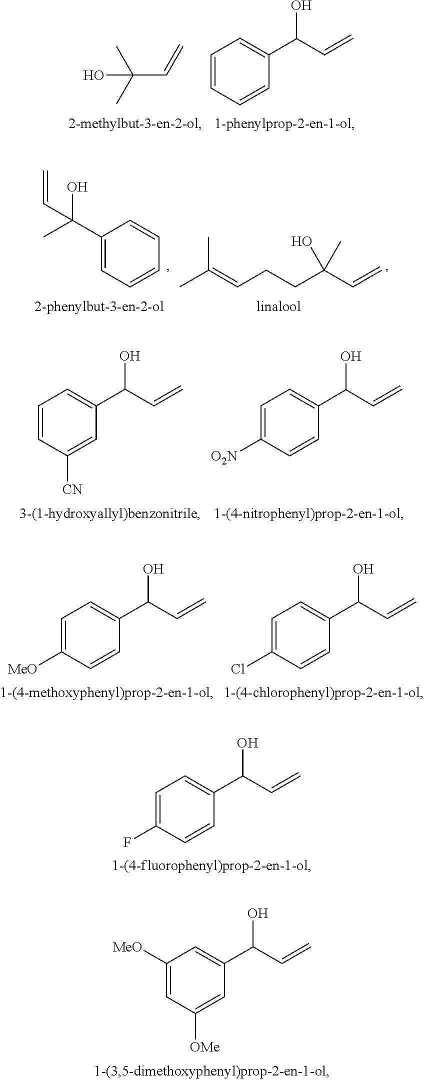

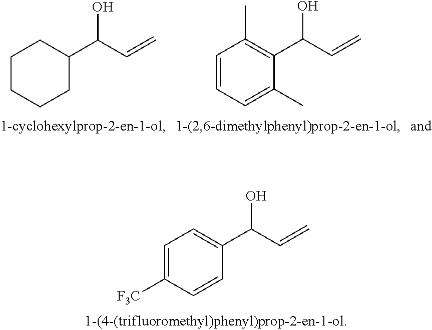

31. (canceled)

32. (canceled)

33. The process of claim 1, wherein the compound of Formula (I) is selected from:

| Compound I.D. | Structure |
|---|---|
| I-1 | 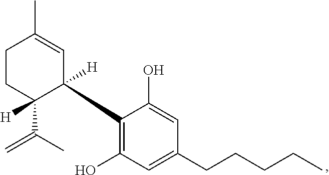 [SMILES: C=C(C)[C@@H]1CCC(C)=C[C@H]1c1c(O)cc(CCCCC)cc1O] |
| I-2 | 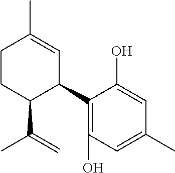 [SMILES: C=C(C)[C@H]1CCC(C)=C[C@H]1c1c(O)cc(C)cc1O] |
| I-3 | 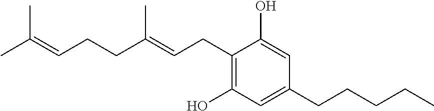 [SMILES: CCCCCc1cc(O)c(C/C=C(\C)CCC=C(C)C)c(O)c1] |
| I-4 | 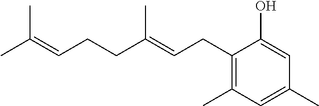 [SMILES: CC(C)=CCC/C(C)=C/Cc1c(C)cc(C)cc1O] |
| I-5 | 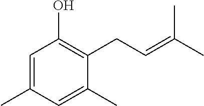 |
| I-6 | 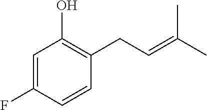 |
| I-7 | 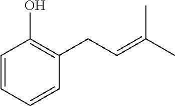 |
| I-8 | 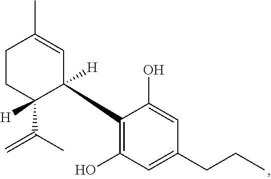 [SMILES: C=C(C)[C@@H]1CCC(C)=C[C@H]1c1c(O)cc(CCC)cc1O] |
| I-9 | 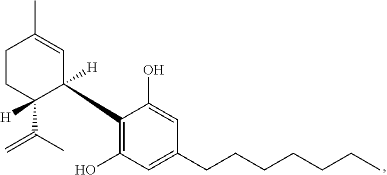 [SMILES: C=C(C)[C@@H]1CCC(C)=C[C@H]1c1c(O)cc(CCCCCCC)cc1O] |
| I-10 | 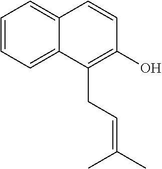 [SMILES: CC(C)=CCc1c(O)ccc2ccccc12] |
| I-11 | 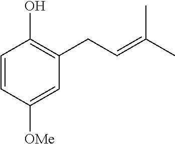 |
| I-12 | 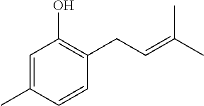 |
| I-13 | 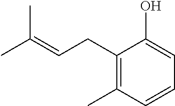 |
| I-14 | 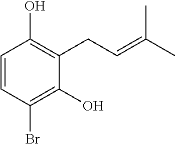 |
| I-15 | 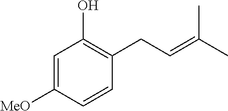 |
| I-16 | 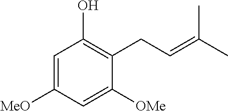 [SMILES: COc1cc(O)c(CC=C(C)C)c(OC)c1] |
| I-17 | 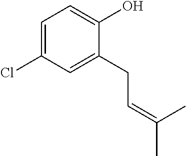 |
| I-18 | 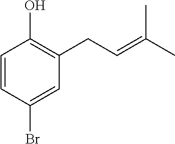 |
| I-19 | 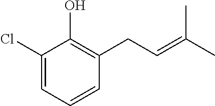 |
| I-20 | 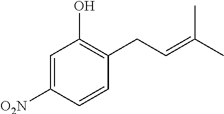 |
| I-21 | 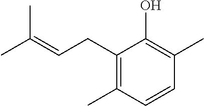 |
| I-22 | 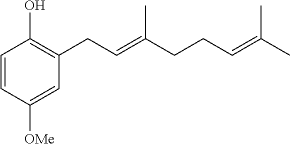 [SMILES: COc1ccc(O)c(C/C=C(\C)CCC=C(C)C)c1] |
| I-23 | 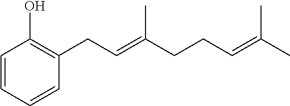 [SMILES: CC(C)=CCC/C(C)=C/Cc1ccccc1O] |
| I-24 | 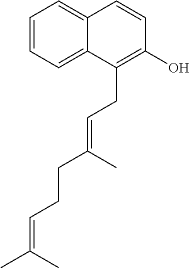 [SMILES: CC(C)=CCC/C(C)=C/Cc1c(O)ccc2ccccc12] |
| I-25 | 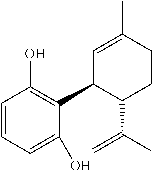 |
| I-26 | 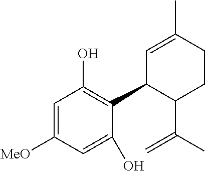 [SMILES: C=C(C)C1CCC(C)=C[C@@H]1c1c(O)cc(OC)cc1O] |
| I-27 | 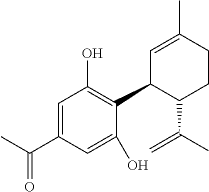 |
| I-28 | 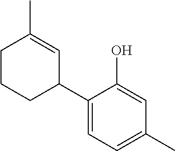 |
| I-29 | 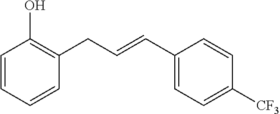 [SMILES: Oc1ccccc1C/C=C/c1ccc(C(F)(F)F)cc1] |
| I-30 | 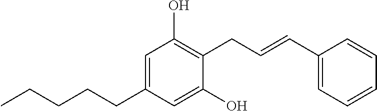 [SMILES: CCCCCc1cc(O)c(C/C=C/c2ccccc2)c(O)c1] |
| I-31 | 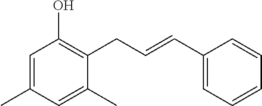 [SMILES: Cc1cc(C)c(C/C=C/c2ccccc2)c(O)c1] |
| I-32 | 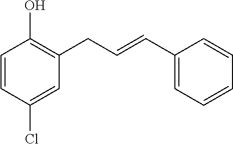 [SMILES: Oc1ccc(Cl)cc1C/C=C/c1ccccc1] |
| I-33 | 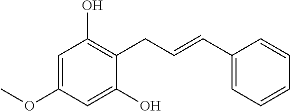 [SMILES: COc1cc(O)c(C/C=C/c2ccccc2)c(O)c1] |
| I-34 | 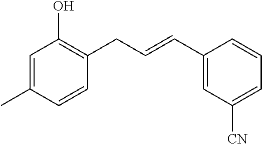 [SMILES: Cc1ccc(C/C=C/c2cccc(C#N)c2)c(O)c1] |
| I-35 | 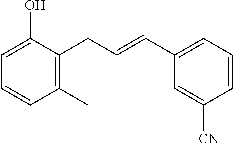 [SMILES: Cc1cccc(O)c1C/C=C/c1cccc(C#N)c1] |
| I-36 | 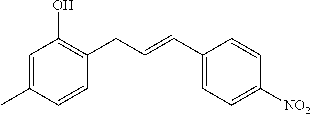 [SMILES: Cc1ccc(C/C=C/c2ccc([N+](=O)[O-])cc2)c(O)c1] |
| I-37 | 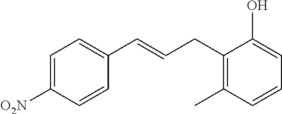 [SMILES: Cc1cccc(O)c1C/C=C/c1ccc([N+](=O)[O-])cc1] |
| I-38 | 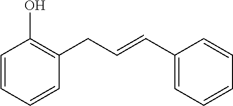 [SMILES: Oc1ccccc1C/C=C/c1ccccc1] |
| I-39 | 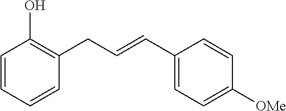 [SMILES: COc1ccc(/C=C/Cc2ccccc2O)cc1] |
| I-40 | 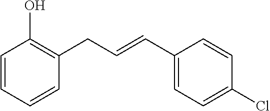 [SMILES: Oc1ccccc1C/C=C/c1ccc(Cl)cc1] |
| I-41 | 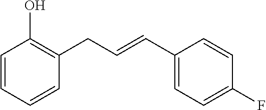 [SMILES: Oc1ccccc1C/C=C/c1ccc(F)cc1] |
| I-42 | 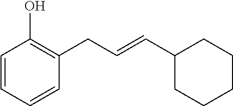 [SMILES: Oc1ccccc1C/C=C/C1CCCCC1] |
| I-43 | 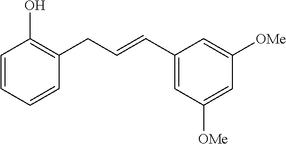 [SMILES: COc1cc(/C=C/Cc2ccccc2O)cc(OC)c1] |
| I-44 | 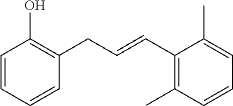 [SMILES: Cc1cccc(C)c1/C=C/Cc1ccccc1O] |
| I-45 | 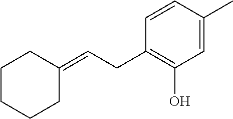 [SMILES: Cc1ccc(CC=C2CCCCC2)c(O)c1] |
| I-46 | 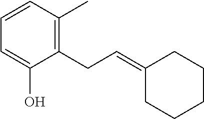 [SMILES: Cc1cccc(O)c1CC=C1CCCCC1] |
| I-47 | 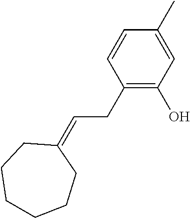 [SMILES: Cc1ccc(CC=C2CCCCCC2)c(O)c1] |
| I-48 | 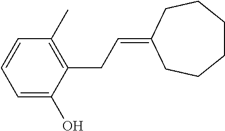 [SMILES: Cc1cccc(O)c1CC=C1CCCCCC1] |
| I-49 | 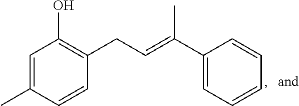 [SMILES: C/C(=C\Cc1ccc(C)cc1O)c1ccccc1] |
| I-50 | 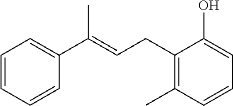 [SMILES: C/C(=C\Cc1c(C)cccc1O)c1ccccc1] |

34. A process for preparing a compound of Formula I-A: 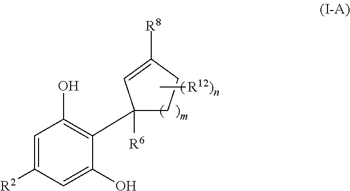
comprising reacting a compound of Formula (II): 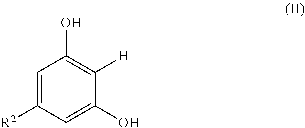
with a compound of Formula (Ill-A): 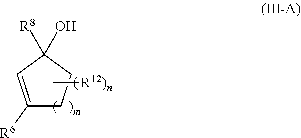
in presence of aluminum compound selected from alumina and aluminum alkoxides and in a non-protic solvent to form the compound of Formula (I-A),
wherein:
R² is selected from H, OH, protected hydroxyl, halo, CN, NO₂, COOH, C₁₋₁₀alkyl, C₁₋₁₀haloalkyl, C₂₋₁₀alkenyl, C₂₋₁₀alkynyl, C₃₋₁₀cycloalkyl, C₃₋₁₀heterocycloalkyl, aryl, C₅₋₁₀heteroaryl, Z—C₁₋₁₀alkyl, Z—C₂₋₁₀alkenyl, Z—C₂₋₁₀alkynyl, Z—C₃₋₁₀cycloalkyl, Z—C₃₋₁₀heterocycloalkyl, Z-aryl, and Z—C₅₋₁₀heteroaryl, wherein the later 14 groups are unsubstituted or substituted with one or more substituents independently selected from ═O, OH, NH₂, halo, NHC₁₋₆alkyl, N(C₁₋₆alkyl)(C₁₋₆alkyl), C₁₋₆alkyl, C₂₋₆alkenyl, C₂₋₆alkynyl, OC₁₋₆alkyl, OC₂₋₆alkenyl, OC₂₋₆alkynyl, SC₁₋₆alkyl, SC₂₋₆alkenyl, SC₂₋₆alkynyl, S(O)C₁₋₆alkyl, S(O)C₂₋₆alkenyl, S(O)C₂₋₆alkynyl, SO₂C₁₋₆alkyl, SO₂C₂₋₆alkenyl, SO₂C₂₋₆alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆haloalkyl, C₂₋₆alkenyl, OC₁₋₆alkyl and OC₁₋₆haloalkyl;
Z is selected from O, C(O), CO₂, S, SO₂, SO, and NR¹⁰;
R⁶ is selected from H, C₁₋₆alkyl and fluoroC₁₋₆alkyl;
R⁸ is selected from H, C₁₋₆alkyl and fluoroC₁₋₆alkyl;
R¹⁰ is selected from C₁₋₆alkyl and C₁₋₆fluoroalkyl;
each R¹² is independently selected from ═O, OH, NH₂, halo, NHC₁₋₁₀alkyl, N(C₁₋₁₀alkyl)(C₁₋₁₀alkyl), C₁₋₁₀alkyl, C₂₋₁₀alkenyl, C₂₋₁₀alkynyl, OC₁₋₁₀alkyl, OC₁₋₁₀fluoroalkyl, OC₂₋₁₀alkenyl, OC₂₋₁₀alkynyl, SC₁₋₁₀alkyl, SC₁₋₁₀fluoroalkyl SC₂₋₁₀alkenyl, SC₂₋₁₀alkynyl, S(O)C₁₋₁₀alkyl, S(O)C₁₋₁₀fluoroalkyl, S(O)C₂₋₁₀alkenyl, S(O)C₂₋₁₀alkynyl, SO₂C₁₋₁₀alkyl, SO₂C₁₋₁₀fluoroalkyl SO₂C₂₋₁₀alkenyl, SO₂C₂₋₁₀alkynyl, aryl, C₅₋₁₀heteroaryl, C₃₋₁₀cycloalkyl, and C₃₋₁₀heterocycloalkyl, the latter 21 groups being unsubstituted or further substituted with one or more substituents independently selected from OH, halo, C₁₋₆alkyl, C₁₋₆fluoroalkyl C₂₋₆alkenyl, OC₁₋₆alkyl and OC₁₋₆fluoroalkyl;
m is 1, 2, 3, 4, 5, 6, 7, or 8; and
n is 1, 2, 3 or 4.

35. The process of claim 1, wherein the process provides compounds of Formula I-B 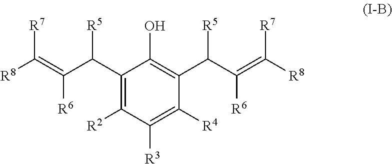
wherein R¹, R², R³, R⁴, R⁵, R⁶, R⁷, R⁸ are as defined in claim 1.

36. The process of claim 35, wherein the compound of Formula (I-B) is selected from the compounds listed below:

| Compound I.D. | Structure |
|---|---|
| I-51 | 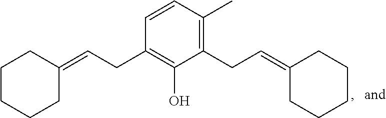 [SMILES: Cc1ccc(CC=C2CCCCC2)c(O)c1CC=C1CCCCC1] |
| I-52 | 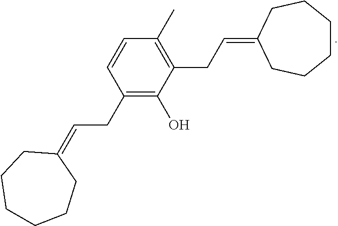 [SMILES: Cc1ccc(CC=C2CCCCCC2)c(O)c1CC=C1CCCCCC1] |

37. The process of claim 1, wherein the aluminum compound is alumina.

38. The process of claim 37, wherein the compound of Formula (I) is formed by reacting the compound of Formula (II) with a compound of Formula (III) in the presence of alumina and further additives and the further additives are dehydrating reagents and/or an acid.

39. (canceled)

40. The process of claim 1, wherein the aluminum compound is an aluminum alkoxide.

41.-43. (canceled)

44. The process of claim 1, wherein the non-protic solvent is selected from hexane, hexanes, heptane, heptanes, cyclohexane, petroleum ether, octane, diglyme, toluene, xylenes, benzene, chloroform, fluorinated alkanes, dichloromethane (DCM), 1,2-dichloroethane (DCE), ethyl acetate, carbon tetrachloride, tetrahydrofuran (THF), diethyl ether, diisopropyl ether, isooctane, methyl ethyl ketone, acetone, dimethyl sulfoxide, dimethylformamide, methyl tert-butyl ether, trichloroethane, n-butyl acetate, chlorobenzene acetonitrile, and trifluorotoluene, and mixtures thereof.

45.-48. (canceled)

49. The process of claim 1, wherein the process comprises mixing the compound of Formula (II), the compound of Formula (III) and the aluminum compound, and any optional additives, in the non-protic solvent with the addition of about 2 to about 4, about 2.5 to about 3.5, or about 3 molar equivalents of the compound of Formula (II) relative to the amount of the compound of Formula (III).

50. (canceled)
